(19) United States
(12) Patent Application Publication
(10) Pub. No.: US 2004/0235005 A1
(43) Pub. Date: Nov. 25, 2004

(54) Methods and composition for detecting targets

(76) Inventors: Ernest Friedlander, San Francisco, CA (US); Shirley J. Johnson, San Francisco, CA (US); Sabine Short, Dublin, CA (US); H. Michael Wenz, Redwood City, CA (US)

Correspondence Address: FINNEGAN, HENDERSON, FARABOW, GARRETT & DUNNER, LLP, 1300 I STREET, NW, WASHINGTON, DC, 20005, US

(21) Appl. No.: 10/693,609

(22) Filed: Oct. 23, 2003

Related U.S. Application Data
(60) Provisional application No. 60/421,035, filed on Oct. 23, 2002.

(30) Foreign Application Priority Data
Oct. 23, 2002 (WO) PCT/US02/33801

(51) Int. Cl. C12Q 1/68; C12P 19/34

(52) U.S. Cl. 435/6; 435/91.2

(57) ABSTRACT

The present invention relates to methods and kits for detecting the presence or absence of (or quantitating) target nucleic acid sequences using ligation and amplification.

This application claims the benefit of U.S. Provisional Application Serial No. 60/421,035, filed Oct. 23, 2002, and PCT International Application No. PCT/US02/33801, filed Oct. 23, 2002, both of which are expressly incorporated by reference herein.

### I. FIELD OF THE INVENTION

The invention relates to methods and compositions for the detection of targets in a sample.

### II. BACKGROUND

The detection of the presence or absence of (or quantity of) one or more target sequences in a sample containing one or more target sequences is commonly practiced. For example, the detection of cancer and many infectious diseases, such as AIDS and hepatitis, routinely includes screening biological samples for the presence or absence of diagnostic nucleic acid sequences. Also, detecting the presence or absence of nucleic acid sequences is often used in forensic science, paternity testing, genetic counseling, and organ transplantation.

An organism's genetic makeup is determined by the genes contained within the genome of that organism. Genes are composed of long strands or deoxyribonucleic acid (DNA) polymers that encode the information needed to make proteins. Properties, capabilities, and traits of an organism often are related to the types and amounts of proteins that are, or are not, being produced by that organism.

A protein can be produced from a gene as follows. First, the DNA of the gene that encodes a protein, for example, protein “X”, is converted into ribonucleic acid (RNA) by a process known as “transcription.” During transcription, a single-stranded complementary RNA copy of the gene is made. Next, this RNA copy, referred to as protein X messenger RNA (mRNA), is used by the cell's biochemical machinery to make protein X, a process referred to as “translation.” Basically, the cell's protein manufacturing machinery binds to the mRNA, “reads” the RNA code, and “translates” it into the amino acid sequence of protein X. In summary, DNA is transcribed to make mRNA, which is translated to make proteins.

The amount of protein X that is produced by a cell often is largely dependent on the amount of protein X mRNA that is present within the cell. The amount of protein X mRNA within a cell is due, at least in part, to the degree to which gene X is expressed. Whether a particular gene is expressed, and if so, to what level, may have a significant impact on the organism.

### III. SUMMARY OF THE INVENTION

In certain embodiments, methods for detecting the presence or absence of at least one target nucleic acid sequence in a sample are provided. In certain embodiments, the method comprises forming a ligation reaction composition comprising the sample, and a ligation probe set for each target nucleic acid sequence. In certain embodiments, the probe set comprises (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion, wherein the 3′ primer-specific portion comprises a sequence. In certain embodiments, the probes in each set are suitable for ligation together when hybridized adjacent to one another on a complementary target sequence.

In certain embodiments, the methods further comprise forming a test composition by subjecting the ligation reaction composition to at least one cycle of ligation, wherein adjacently hybridizing complementary probes are ligated to one another to form a ligation product comprising the 5′ primer-specific portion, the target-specific portions, and the 3′ primer-specific portion. In certain embodiments, the methods further comprise forming at least one amplification reaction composition comprising:

at least a portion of the test composition;

a polymerase;

a double-stranded-dependent specific label, wherein the double-stranded dependent label has a first detectable signal value when the double-stranded-dependent label is not exposed to double-stranded nucleic acid; and

at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product.

In certain embodiments, the methods further comprise subjecting the at least one amplification reaction composition to at least one amplification reaction. In certain embodiments, the methods further comprise detecting a second detectable signal value at least one of during and after the at least one amplification reaction, wherein a threshold difference between the first detectable signal value and the second detectable signal value indicates the presence of the target nucleic acid sequence, and wherein no threshold difference between the first detectable signal value and the second detectable signal value indicates the absence of the target nucleic acid sequence.

In certain embodiments, methods for detecting the presence or absence of at least one target nucleic acid sequence in a sample are provided. In certain embodiments, the method comprises forming a ligation reaction composition comprising the sample, and a ligation probe set for each target nucleic acid sequence. In certain embodiments, the probe set comprises (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion, wherein the 3′ primer-specific portion comprises a sequence. In certain embodiments, the probes in each set are suitable for ligation together when hybridized adjacent to one another on a complementary target sequence.

In certain embodiments, the methods further comprise forming a test composition by subjecting the ligation reaction composition to at least one cycle of ligation, wherein adjacently hybridizing complementary probes are ligated to one another to form a ligation product comprising the 5′ primer-specific portion, the target-specific portions, and the 3′ primer-specific portion. In certain embodiments, the methods further comprise forming at least one amplification reaction composition comprising:

at least a portion of the test composition;

a polymerase;

a double-stranded-dependent specific label; and

at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product.

In certain embodiments, the methods further comprise subjecting the at least one amplification reaction composition to at least one amplification reaction. In certain embodiments, the methods further comprise detecting the presence or absence of the target nucleic acid sequence by monitoring a signal at least one of during and after the at least one amplification reaction.

In certain embodiments, methods for detecting the presence or absence of at least one target nucleic acid sequence in a sample are provided. In certain embodiments, the method comprises forming at least one reaction composition comprising:

the sample;

a ligation probe set for the target nucleic acid sequence, the probe set comprising (a) at least one first probe, comprising a target-specific portion and a 5′ primer-specific portion, wherein the 5′ primer-specific portion comprises a sequence and (b) at least one second probe, comprising a target-specific portion and a 3′ primer-specific portion, wherein the 3′ primer-specific portion comprises a sequence, wherein the probes in each set are suitable for ligation together when hybridized adjacent to one another on a complementary target sequence;

a polymerase;

a double-stranded-dependent label, wherein the double-stranded-dependent label has a first detectable signal value when the double-stranded-dependent label is not exposed to double-stranded nucleic acid; and

at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product.

In certain embodiments, the methods further comprise subjecting the reaction composition to at least one cycle of ligation, wherein adjacently hybridizing complementary probes are ligated to one another to form a ligation product comprising the 5′ primer-specific portion, the target-specific portions, and the 3′ primer-specific portion.

In certain embodiments, the methods further comprise, after the at least one cycle of ligation, subjecting the reaction composition to at least one amplification reaction. In certain embodiments, the methods further comprise detecting a second detectable signal value at least one of during and after the at least one amplification reaction, wherein a threshold difference between the first detectable signal value and the second detectable signal value indicates the presence of the target nucleic acid sequence, and wherein no threshold difference between the first detectable signal value and the second detectable signal value indicates the absence of the target nucleic acid sequence.

In certain embodiments, methods for detecting the presence or absence of at least one target nucleic acid sequence in a sample are provided. In certain embodiments, the method comprises forming at least one reaction composition comprising:

the sample;

a ligation probe set for the target nucleic acid sequence, the probe set comprising (a) at least one first probe, comprising a target-specific portion and a 5′ primer-specific portion, wherein the 5′ primer-specific portion comprises a sequence and (b) at least one second probe, comprising a target-specific portion and a 3′ primer-specific portion, wherein the 3′ primer-specific portion comprises a sequence, wherein the probes in each set are suitable for ligation together when hybridized adjacent to one another on a complementary target sequence;

a polymerase;

a double-stranded-dependent label; and

at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product.

In certain embodiments, the methods further comprise subjecting the reaction composition to at least one cycle of ligation, wherein adjacently hybridizing complementary probes are ligated to one another to form a ligation product comprising the 5′ primer-specific portion, the target-specific portions, and the 3′ primer-specific portion.

In certain embodiments, the methods further comprise, after the at least one cycle of ligation, subjecting the reaction composition to at least one amplification reaction. In certain embodiments, the methods further comprise detecting the presence or absence of the target nucleic acid sequence by monitoring a signal at least one of during and after the at least one amplification reaction.

In certain embodiments, kits for detecting at least one target nucleic acid sequence in a sample are provided. In certain embodiments, the kits comprise:

(a) a ligation probe set for each target nucleic acid sequence, the probe set comprising

(i) at least one first probe, comprising a target-specific portion, a 5′ primer-specific portion, wherein the 5′ primer-specific portion comprises a sequence, and

(ii) at least one second probe, comprising a target-specific portion, a 3′ primer-specific portion, wherein the 3′ primer-specific portion comprises a sequence,

wherein the probes in each set are suitable for ligation together when hybridized adjacent to one another on a complementary target nucleic acid sequence; and

(b) a double-stranded-dependent label.

In certain embodiments, methods for detecting the presence or absence of at least one target nucleic acid sequence in a sample are provided. In certain embodiments, the method comprises forming a ligation reaction composition comprising the sample, a ligation probe set for each target nucleic acid sequence, and poly-deoxy-inosinic-deoxy-cytidylic acid. In certain embodiments, the probe set comprises (a) at least one first probe, comprising a target-specific portion, and (b) at least one second probe, comprising a target-specific portion, wherein the probes in each set are suitable for ligation together when hybridized adjacent to one another on a complementary target sequence.

In certain embodiments, the methods further comprise forming a test composition by subjecting the ligation reaction composition to at least one cycle of ligation, wherein adjacently hybridizing complementary probes are ligated to one another to form a ligation product comprising the 5′ primer-specific portion, the target-specific portions, and the 3′ primer-specific portion. In certain embodiments, the methods further comprise detecting the presence or absence of the ligation product to detect the presence or absence of the at least one target nucleic acid sequence.

In certain embodiments, methods for detecting the presence or absence of at least one target nucleic acid sequence in a sample are provided. In certain embodiments, the method comprises forming a ligation reaction composition comprising the sample, a ligation probe set for each target nucleic acid sequence, and poly-deoxy-inosinic-deoxy-cytidylic acid. In certain embodiments, the probe set comprises (a) at least one first probe, comprising a target-specific portion, and (b) at least one second probe, comprising a target-specific portion, wherein the probes in each set are suitable for ligation together when hybridized adjacent to one another on a complementary target sequence.

In certain embodiments, the methods further comprise forming a test composition by subjecting the ligation reaction composition to at least one cycle of ligation, wherein adjacently hybridizing complementary probes are ligated to one another to form a ligation product comprising the 5′ primer-specific portion, the target-specific portions, and the 3′ primer-specific portion. In certain embodiments, the methods further comprise forming at least one amplification reaction composition comprising:

at least a portion of the test composition;

a polymerase; and

at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product.

In certain embodiments, the methods further comprise subjecting the at least one amplification reaction composition to at least one amplification reaction. In certain embodiments, the methods further comprise detecting the presence or absence of the target nucleic acid sequence by detecting whether the at least one amplification reaction results in amplification product from ligation product.

In certain embodiments, kits for detecting at least one target nucleic acid sequence in a sample are provided. In certain embodiments, the kits comprise:

(a) a ligation probe set for each target nucleic acid sequence, the probe set comprising

(i) at least one first probe, comprising a target-specific portion, a 5′ primer-specific portion, wherein the 5′ primer-specific portion comprises a sequence, and

(ii) at least one second probe, comprising a target-specific portion, a 3′ primer-specific portion, wherein the 3′ primer-specific portion comprises a sequence,

wherein the probes in each set are suitable for ligation together when hybridized adjacent to one another on a complementary target nucleic acid sequence; and

(b) a buffer comprising poly-deoxy-inosinic-deoxy-cytidylic acid.

In certain embodiments, compositions for a ligation reaction comprising a ligase and poly-deoxy-inosinic-deoxy-cytidylic acid are provided.

### IV. BRIEF DESCRIPTION OF THE DRAWINGS

The skilled artisan will understand that the drawings, described below, are for illustration purposes only. The figures are not intended to limit the scope of the invention in any way.

FIG. 1 is a schematic showing a ligation probe set according to certain embodiments of the invention.

Each probe includes a portion that is complementary to the target (the “target-specific portion,” T-SP) and a portion that is complementary to or has the same sequence as a primer (the “primer-specific portion,” P-SP). Each probe set comprises at least one first probe and at least one second probe that are designed to hybridize with the target with the 3′ end of the first probe immediately adjacent to and opposing the 5′ end of the second probe.

FIG. 2 is a schematic showing an exemplary embodiment of certain embodiments comprising ligation and primer extension amplification.

FIG. 3 depicts a method for differentiating between two potential alleles in a target locus using certain embodiments of the invention.

FIG. 3(A) shows: (i) a target-specific probe set comprising: two first probes (A and B) that have the same target-specific portions except for different pivotal complements (here, T at the 3′ end probe A and C at the 3′ end probe B) and different primer-specific portions ((P-SPA) and (P-SPB)); and one second probe (Z) comprising a target-specific portion and a primer-specific portion (P-SP2).

FIG. 3(B) shows the three probes annealed to the target. The target-specific portion of probe A is fully complementary with the 3′ target region including the pivotal nucleotide. The pivotal complement of probe B is not complementary with the 3′ target region. The target-specific portion of probe B, therefore, contains a base-pair mismatch at the 3′ end. The target-specific portion of probe Z is fully complementary to the 5′ target region.

FIG. 3(C) shows ligation of probes A and Z to form ligation product A-Z. Probes B and Z are not ligated together to form a ligation product due to the mismatched pivotal complement on probe B.

FIG. 3(D) shows denaturing the double-stranded molecules to release the A-Z ligation product and unligated probes B and Z.

FIG. 4 depicts certain embodiments employing flap endonuclease.

FIG. 5 depicts certain embodiments employing flap endonuclease.

FIG. 6 depicts certain embodiments employing flap endonuclease.

FIG. 7 depicts certain embodiments employing flap endonuclease.

FIG. 8 is a schematic depicting certain embodiments of the invention.

FIG. 8(A) depicts a target sequence and a ligation probe set comprising: two first probes (A and B) that have the same target-specific portions except for different pivotal complements (here, T at the 3′ end probe A and G at the 3′ end probe B) and different primer-specific portions ((P-SPA) and (P-SPB)); and one second probe (Z) comprising a target-specific portion and a primer-specific portion (P-SP2).

FIG. 8(B) depicts the A and Z probes hybridized to the target sequence under annealing conditions.

FIG. 8(C) depicts the ligation of the first and second probes in the presence of a ligation agent to form ligation product.

FIG. 8(D) depicts denaturing the ligation product:target complex to release a single-stranded ligation product; and performing two separate amplification reactions with either primer set (PA) and (P2) or primer set (PB) and (P2).

FIG. 9 depicts certain embodiments involving three biallelic loci.

FIG. 10 depicts certain embodiments involving three biallelic loci.

FIG. 11 depicts certain embodiments in which one probe of a ligation probe set also serves as a primer.

FIG. 12 depicts exemplary alternative splicing.

FIG. 13 depicts certain embodiments involving splice variants.

FIG. 14 relates to certain embodiments employing ΔCₜ values.

### V. DETAILED DESCRIPTION OF CERTAIN EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”,as well as other forms, such as “includes” and “included”, is not limiting. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. U.S. patent application Ser. Nos. 09/584,905, filed May 30, 2000, 09/724,755, filed Nov. 28, 2000, 10/011,993, filed Dec. 5, 2001, and 60/412,225, filed Sep. 19, 2002, and Patent Cooperation Treaty Application No. PCT/US01/17329, filed May 30, 2001, are hereby expressly incorporated by reference in their entirety for any purpose.

A. Certain Definitions

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-A2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylctosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”,as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′-linked and 3′-4′-linked and other “locked” or “LNA”,bicyclic sugar modifications (see, e.g., PCT published application nos. WO 98/22489, WO 98/39352; and WO 99/14226). Exemplary LNA sugar analogs within a polynucleotide include, but are not limited to, the structures:

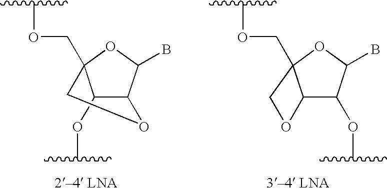

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2nd Ed., Freeman, San Francisco, Calif.).

One or more of the pentose carbons of a nucleotide may be substituted with a phosphate ester having the formula:

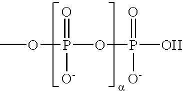

where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

The term “nucleotide analog”, as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers that can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

As used herein, the terms “polynucleotide”, “oligonucleotide”, and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄ ⁺, trialkylammonium, Mg^{2 +}, Na⁺ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. nucleic acids typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, “T” denotes thymidine or an analog thereof, and “U” denotes uridine or an analog thereof, unless otherwise noted.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below:

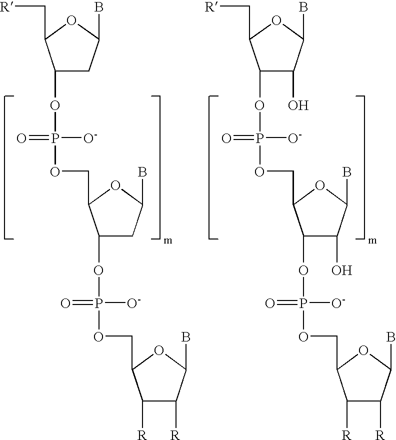

wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl or (C5-C14) aryl, or two adjacent Rs are taken together to form a bond such that the ribose sugar is 2′,3′-didehydroribose; and each R′ is independently hydroxyl or

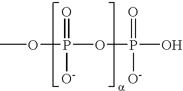

where α is zero, one or two.

In certain embodiments of the ribopolynucleotides and 2′-dexyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254:1497-1500; WO 92/20702; U.S. Pat. No. 5,719,262; U.S. Pat. No. 5,698,685; ); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

An “enzymatically active mutant or variant thereof,” when used in reference to an enzyme such as a polymerase or a ligase, means a protein with appropriate enzymatic activity. Thus, for example, but without limitation, an enzymatically active mutant or variant of a DNA polymerase is a protein that is able to catalyze the stepwise addition of appropriate deoxynucleoside triphosphates into a nascent DNA strand in a template-dependent manner. An enzymatically active mutant or variant differs from the “generally-accepted” or consensus sequence for that enzyme by at least one amino acid, including, but not limited to, substitutions of one or more amino acids, addition of one or more amino acids, deletion of one or more amino acids, and alterations to the amino acids themselves. With the change, however, at least some catalytic activity is retained. In certain embodiments, the changes involve conservative amino acid substitutions. Conservative amino acid substitution may involve replacing one amino acid with another that has, e.g., similar hydrophobicity, hydrophilicity, charge, or aromaticity. In certain embodiments, conservative amino acid substitutions may be made on the basis of similar hydropathic indices. A hydropathic index takes into account the hydrophobicity and charge characteristics of an amino acid, and in certain embodiments, may be used as a guide for selecting conservative amino acid substitutions. The hydropathic index is discussed, e.g., in Kyte et al., J. Mol. Biol., 157:105-131 (1982). It is understood in the art that conservative amino acid substitutions may be made on the basis of any of the aforementioned characteristics.

Alterations to the amino acids may include, but are not limited to, glycosylation, methylation, phosphorylation, biotinylation, and any covalent and noncovalent additions to a protein that do not result in a change in amino acid sequence. “Amino acid” as used herein refers to any amino acid, natural or non-natural, that may be incorporated, either enzymatically or synthetically, into a polypeptide or protein.

Fragments, for example, but without limitation, proteolytic cleavage products, are also encompassed by this term, provided that atleast some enzyme catalytic activity is retained.

The skilled artisan will readily be able to measure catalytic activity using an appropriate well-known assay. Thus, an appropriate assay for polymerase catalytic activity might include, for example, measuring the ability of a variant to incorporate, under appropriate conditions, rNTPs or dNTPs into a nascent polynucleotide strand in a template-dependent manner. Likewise, an appropriate assay for ligase catalytic activity might include, for example, the ability to ligate adjacently hybridized oligonucleotides comprising appropriate reactive groups. Protocols for such assays may be found, among other places, in Sambrook et al., Molecular Cloning, A Laboratory Manual, Cold Spring Harbor Press (1989) (hereinafter “Sambrook et al.”), Sambrook and Russell, Molecular Cloning, Third Edition, Cold Spring Harbor Press (2000) (hereinafter “Sambrook and Russell”), Ausbel et al., Current Protocols in Molecular Biology (1993) including supplements through April 2001, John Wiley & Sons (hereinafter “Ausbel et al.”).

A “target” or “target nucleic acid sequence” according to the present invention comprises a specific nucleic acid sequence that can be distinguished by a probe. Targets may include both naturally occurring and synthetic molecules.

“Probes”, according to the present invention, comprise oligonucleotides that comprise a specific portion that is designed to hybridize in a sequence-specific manner with a complementary region on a specific nucleic acid sequence, e.g., a target nucleic acid sequence. In certain embodiments, the specific portion of the probe may be specific for a particular sequence, or alternatively, may be degenerate, e.g., specific for a set of sequences.

A “ligation probe set” according to the present invention is a group of two or more probes designed to detect at least one target. As a non-limiting example, a ligation probe set may comprise two nucleic acid probes designed to hybridize to a target such that, when the two probes are hybridized to the target adjacent to one another, they are suitable for ligation together.

When used in the context of the present invention, “suitable for ligation” refers to at least one first target-specific probe and at least one second target-specific probe, each comprising an appropriately reactive group. Exemplary reactive groups include, but are not limited to, a free hydroxyl group on the 3′ end of the first probe and a free phosphate group on the 5′ end of the second probe. In certain embodiments, the second probe may be a 5′-adenylated probe, in which the 5′-phosphate of adenosine is attached to the 5′ end of the probe (a phosphoanhydride linkage). Exemplary pairs of reactive groups include, but are not limited to: phosphorothioate and tosylate or iodide; esters and hydrazide; RC(O)S⁻, haloalkyl, or RCH₂S and α-haloacyl; thiophosphoryl and bromoacetoamido groups. Exemplary reactive groups include, but are not limited to, S-pivaloyloxymethyl-4-thiothymidine. Additionally, in certain embodiments, first and second target-specific probes are hybridized to the target sequence such that the 3′ end of the first target-specific probe and the 5′ end of the second target-specific probe are immediately adjacent to allow ligation.

The term “detectable signal value” refers to a value of the signal that is detected from a label. In certain embodiments, the detectable signal value is the amount or intensity of signal that is detected from a label. Thus, if there is no detectable signal from a label, its detectable signal value is zero (0). In certain embodiments, the detectable signal value is a characteristic of the signal other than the amount or intensity of the signal, such as the spectra, wavelength, color, or lifetime of the signal.

“Detectably different signal value” means that one or more detectable signal values are distinguishable from one another by at least one detection method.

The term “double-stranded-dependent label” refers to a label that provides a delectably different signal value when it is exposed to double-stranded nucleic acid than when it is not exposed to double-stranded nucleic acid.

The term “threshold difference between detectable signal values” refers to a set difference between a first detectable signal value and a second detectable signal value that results when the target nucleic acid sequence that is being sought is present in a sample, but that does not result when the target nucleic acid sequence is absent. The first detectable signal value of a double-stranded-dependent label is the detectable signal value from the label when it is not exposed to double-stranded nucleic acid. The second detectable signal value is detected during and/or after an amplification reaction using a composition that comprises the double-stranded-dependent label.

The term “quantitating,” when used in reference to an amplification product, refers to determining the quantity or amount of a particular sequence that is representative of a target nucleic acid sequence in the sample. For example, but without limitation, one may measure the intensity of the signal from a label. The intensity or quantity of the signal is typically related to the amount of amplification product. The amount of amplification product generated correlates with the amount of target nucleic acid sequence present prior to ligation and amplification, and thus, in certain embodiments, may indicate the level of expression for a particular gene.

The term “amplification product” as used herein refers to the product of an amplification reaction including, but not limited to, primer extension, the polymerase chain reaction (PCR), RNA transcription, and the like. Thus, exemplary amplification products may comprise at least one of primer extension products, PCR amplicons, RNA transcription products, and the like.

“Primers” according to the present invention refer to oligonucleotides that are designed to hybridize with the primer-specific portion of probes, ligation products, or amplification products in a sequence-specific manner, and serve as primers for amplification reactions.

A “universal primer” is capable of hybridizing to the primer-specific portion of more than one species of probe, ligation product, or amplification product, as appropriate. A “universal primer set” comprises a first primer and a second primer that hybridize with a plurality of species of probes, ligation products, or amplification products, as appropriate.

A “ligation agent” according to the present invention may comprise any number of enzymatic or chemical (i.e., non-enzymatic) agents that can effect ligation of nucleic acids to one another.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, polynucleotides, oligonucleotides, probes, primers, primer-specific portions, and target-specific portions.

In this application, a statement that one sequence is complementary to another sequence encompasses situations in which the two sequences have mismatches. Here, the term “sequence” encompasses, but is not limited to, nucleic acid sequences, polynucleotides, oligonucleotides, probes, primers, primer-specific portions, and target-specific portions. Despite the mismatches, the two sequences should selectively hybridize to one another under appropriate conditions.

The term “selectively hybridize” means that, for particular identical sequences, a substantial portion of the particular identical sequences hybridize to a given desired sequence or sequences, and a substantial portion of the particular identical sequences do not hybridize to other undesired sequences. A “substantial portion of the particular identical sequences” in each instance refers to a portion of the total number of the particular identical sequences, and it does not refer to a portion of an individual particular identical sequence. In certain embodiments, “a substantial portion of the particular identical sequences” means at least 90% of the particular identical sequences. In certain embodiments, “a substantial portion of the particular identical sequences” means at least 95% of the particular identical sequences.

In certain embodiments, the number of mismatches that may be present may vary in view of the complexity of the composition. Thus, in certain embodiments, fewer mismatches may be tolerated in a composition comprising DNA from an entire genome than a composition in which fewer DNA sequences are present. For example, in certain embodiments, with a given number of mismatches, a probe may more likely hybridize to undesired sequences in a composition with the entire genomic DNA than in a composition with fewer DNA sequences, when the same hybridization conditions are employed for both compositions. Thus, that given number of mismatches may be appropriate for the composition with fewer DNA sequences, but fewer mismatches may be more optimal for the composition with the entire genomic DNA.

In certain embodiments, sequences are complementary if they have no more than 20% mismatched nucleotides. In certain embodiments, sequences are complementary if they have no more than 15% mismatched nucleotides. In certain embodiments, sequences are complementary if they have no more than 10% mismatched nucleotides. In certain embodiments, sequences are complementary if they have no more than 5% mismatched nucleotides.

In this application, a statement that one sequence hybridizes or binds to another sequence encompasses situations where the entirety of both of the sequences hybridize or bind to one another, and situations where only a portion of one or both of the sequences hybridizes or binds to the entire other sequence or to a portion of the other sequence. Here, the term “sequence” encompasses, but is not limited to, nucleic acid sequences, polynucleotides, oligonucleotides, probes, primers, primer-specific portions, and target-specific portions.

In certain embodiments, the term “to a measurably lesser extent” encompasses situations in which the event in question is reduced at least 10 fold. In certain embodiments, the term “to a measurably lesser extent” encompasses situations in which the event in question is reduced at least 100 fold.

In certain embodiments, a statement that a component may be, is, or has been “substantially removed” means that at least 90% of the component may be, is, or has been removed. In certain embodiments, a statement that a component may be, is, or has been “substantially removed” means that at least 95% of the component may be, is, or has been removed.

B. Certain Components

In certain embodiments, target nucleic acid sequences may include RNA and DNA. Exemplary RNA target sequences include, but are not limited to, mRNA, rRNA, tRNA, viral RNA, and variants of RNA, such as splicing variants. Exemplary DNA target sequences include, but are not limited to, genomic DNA, plasmid DNA, phage DNA, nucleolar DNA, mitochondrial DNA, and chloroplast DNA.

In certain embodiments, target nucleic acid sequences include, but are not limited to, cDNA, yeast artificial chromosomes (YAC's), bacterial artificial chromosomes (BAC's), other extrachromosomal DNA, and nucleic acid analogs. Exemplary nucleic acid analogs include, but are not limited to, LNAs, PNAs, PPG's, and other nucleic acid analogs. PPG is pyrrazolopyrimidine dG, which is discussed, e.g., in Sedelnikova et al., Antisense Nucleic Acid Drug Dev 2000, 10(6):443-452 (December 2000).

A variety of methods are available for obtaining a target nucleic acid sequence for use with the compositions and methods of the present invention. When the nucleic acid target is obtained through isolation from a biological matrix, certain isolation techniques include, but are not limited to, (1) organic extraction followed by ethanol precipitation, e.g., using a phenol/chloroform organic reagent (e.g., Ausubel et al., eds., Current Protocols in Molecular Biology Volume 1, Chapter 2, Section I, John Wiley & Sons, New York (1993)), in certain embodiments, using an automated DNA extractor, e.g., the Model 341 DNA Extractor available from Applied Biosystems (Foster City, Calif.); (2) stationary phase adsorption methods (e.g., Boom et al., U.S. Pat. No. 5,234,809; Walsh et al., Biotechniques 10(4): 506-513 (1991)); and (3) salt-induced DNA precipitation methods (e.g., Miller et al., Nucleic Acids Research,16(3): 9-10 (1988)), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. patent application Ser. No. 09/724,613.

In certain embodiments, a target nucleic acid sequence may be derived from any living, or once living, organism, including but not limited to prokaryote, eukaryote, plant, animal, and virus. In certain embodiments, the target nucleic acid sequence may originate from a nucleus of a cell, e.g., genomic DNA, or may be extranuclear nucleic acid, e.g., plasmid, mitrochondrial nucleic acid, various RNAs, and the like. In certain embodiments, if the sequence from the organism is RNA, it may be reverse-transcribed into a cDNA target nucleic acid sequence. Furthermore, in certain embodiments, the target nucleic acid sequence may be present in a double-stranded or single stranded form.

Exemplary target nucleic acid sequences include, but are not limited to, amplification products, ligation products, transcription products, reverse transcription products, primer extension products, methylated DNA, and cleavage products. Exemplary amplification products include, but are not limited to, PCR and isothermal products.

In certain embodiments, nucleic acids in a sample may be subjected to a cleavage procedure. In certain embodiments, such cleavage products may be targets.

Different target nucleic acid sequences may be different portions of a single contiguous nucleic acid or may be on different nucleic acids. Different portions of a single contiguous nucleic acid may or may not overlap.

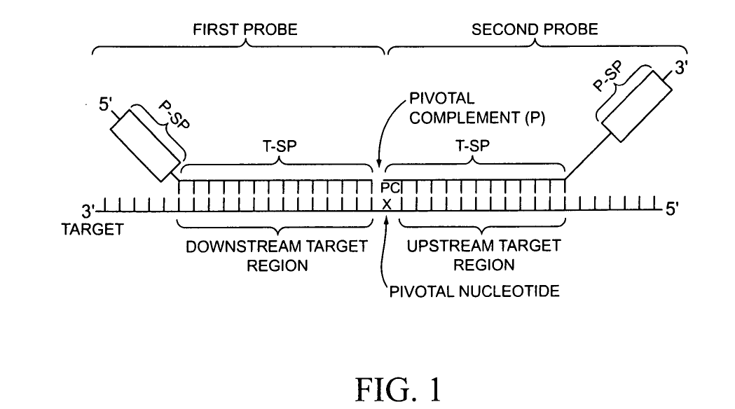

In certain embodiments, a target nucleic acid sequence comprises an upstream or 5′ region, a downstream or 3′ region, and a “pivotal nucleotide” located in the upstream region or the downstream region (see, e.g., FIG. 1). In certain embodiments, the pivotal nucleotide may be the nucleotide being detected by the probe set and may represent, for example, without limitation, a single polymorphic nucleotide in a multiallelic target locus. In certain embodiments, more than one pivotal nucleotide is present. In certain embodiments, one or more pivotal nucleotides is located in the upstream region, and one or more pivotal nucleotide is located in the downstream region. In certain embodiments, more than one pivotal nucleotide is located in the upstream region or the downstream region.

The person of ordinary skill will appreciate that while a target nucleic acid sequence is typically described as a single-stranded molecule, the opposing strand of a double-stranded molecule comprises a complementary sequence that may also be used as a target sequence.

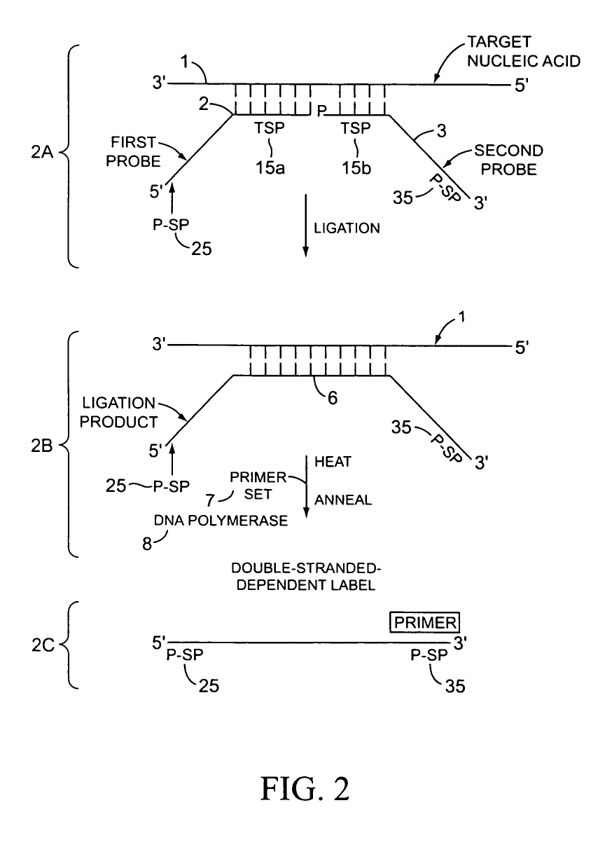

A ligation probe set, according to certain embodiments, comprises two or more probes that comprise a target-specific portion that is designed to hybridize in a sequence-specific manner with a complementary region on a specific target nucleic acid sequence (see, e.g., probes 2 and 3 in FIG. 2). A probe of a ligation probe set may further comprise a primer-specific portion. In certain embodiments, any of the probe's components may overlap any other probe component(s). For example, but without limitation, the target-specific portion may overlap the primer-specific portion.

The sequence-specific portions of probes are of sufficient length to permit specific annealing to complementary sequences in primers and targets as appropriate. In certain embodiments, the length of the primer-specific portions are any number of nucleotides from 6 to 35. In certain embodiments, the length of the target-specific portions are any number of nucleotides from 6 to 35. Detailed descriptions of probe design that provide for sequence-specific annealing can be found, among other places, in Diffenbach and Dveksler, PCR Primer, A Laboratory Manual, Cold Spring Harbor Press, 1995, and Kwok et al., Nucl. Acid Res. 18:999-1005 (1990).

A ligation probe set according to certain embodiments comprises at least one first probe and at least one second probe that adjacently hybridize to the same target nucleic acid sequence. According to certain embodiments, a ligation probe set is designed so that the target-specific portion of the first probe will hybridize with the downstream target region (see, e.g., probe 2 in FIG. 2) and the target-specific portion of the second probe will hybridize with the upstream target region (see, e.g., probe 3 in FIG. 2). The sequence-specific portions of the probes are of sufficient length to permit specific annealing with complementary sequences in targets and primers, as appropriate.

Under appropriate conditions, adjacently hybridized probes may be ligated together to form a ligation product, provided that they comprise appropriate reactive groups, for example, without limitation, a free 3′-hydroxyl and 5′-phosphate group.

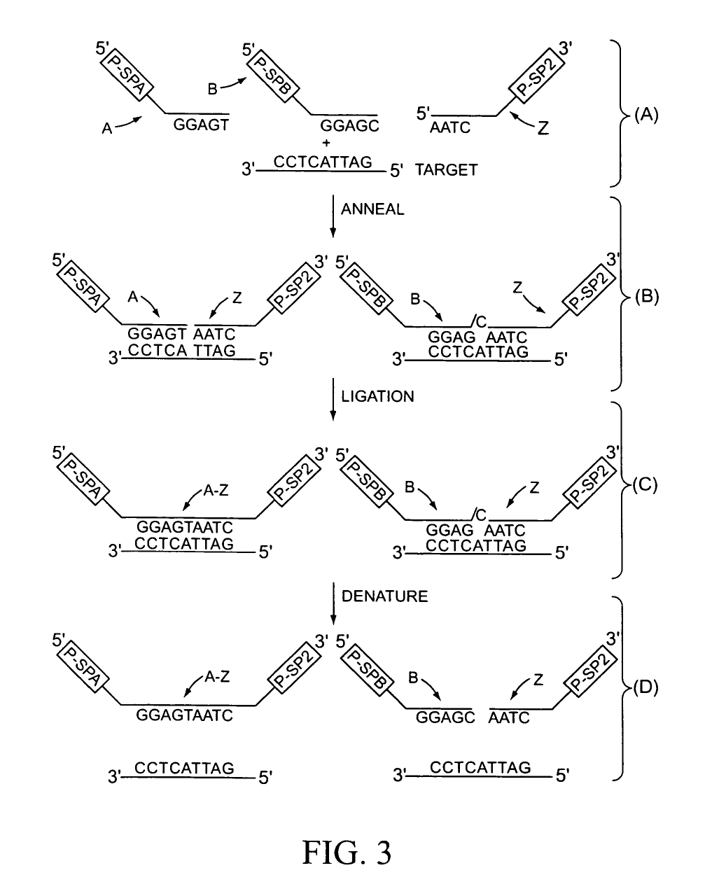

According to certain embodiments, some ligation probe sets may comprise more than one first probe or more than one second probe to allow sequence discrimination between target sequences that differ by one or more nucleotides (see, e.g., FIG. 3).

According to certain embodiments of the invention, a ligation probe set is designed so that the target-specific portion of the first probe will hybridize with the downstream target region (see, e.g., the first probe in FIG. 1) and the target-specific portion of the second probe will hybridize with the upstream target region (see, e.g., the second probe in FIG. 1). In certain embodiments, a nucleotide base complementary to the pivotal nucleotide, the “pivotal complement” or “pivotal complement nucleotide,” is present on the proximal end of the second probe of the target-specific probe set (see, e.g., 5′ end (PC) of the second probe in FIG. 1). In certain embodiments, the first probe may comprise the pivotal complement rather than the second probe (see, e.g., FIG. 3). The skilled artisan will appreciate that, in various embodiments, the pivotal nucleotide(s) may be located anywhere in the target sequence and that likewise, the pivotal complement(s) may be located anywhere within the target-specific portion of the probe(s). For example, according to various embodiments, the pivotal complement may be located at the 3′ end of a probe, at the 5′ end of a probe, or anywhere between the 3′ end and the 5′ end of a probe.

In certain embodiments, when the first and second probes of the ligation probe set are hybridized to the appropriate upstream and downstream target regions, and when the pivotal complement is at the 5′ end of one probe or the 3′ end of the other probe, and the pivotal complement is base-paired with the pivotal nucleotide on the target sequence, the hybridized first and second probes may be ligated together to form a ligation product (see, e.g., FIG. 3(B)-(C)). In the example shown in FIG. 3(B)-(C), a mismatched base at the pivotal nucleotide, however, interferes with ligation, even if both probes are otherwise fully hybridized to their respective target regions.

In certain embodiments, other mechanisms may be employed to avoid ligation of probes that do not include the correct complementary nucleotide at the pivotal complement. For example, in certain embodiments, conditions may be employed such that a probe of a ligation probe set will hybridize to the target sequence to a measurably lesser extent if there is a mismatch at the pivotal nucleotide. Thus, in such embodiments, such non-hybridized probes will not be ligated to the other probe in the probe set.

In certain embodiments, the first probes and second probes in a ligation probe set are designed with similar melting temperatures (Tₘ). Where a probe includes a pivotal complement, in certain embodiments, the Tₘ for the probe(s) comprising the pivotal complement(s) of the target pivotal nucleotide sought will be approximately 4-15° C. lower than the other probe(s) that do not contain the pivotal complement in the probe set. In certain such embodiments, the probe comprising the pivotal complement(s) will also be designed with a Tₘ near the ligation temperature. Thus, a probe with a mismatched nucleotide will more readily dissociate from the target at the ligation temperature. The ligation temperature, therefore, in certain embodiments provides another way to discriminate between, for example, multiple potential alleles in the target.

Further, in certain embodiments, ligation probe sets do not comprise a pivotal complement at the terminus of the first or the second probe (e.g., at the 3′ end or the 5′ end of the first or second probe). Rather, the pivotal complement is located somewhere between the 5′ end and the 3′ end of the first or second probe. In certain such embodiments, probes with target-specific portions that are fully complementary with their respective target regions will hybridize under high stringency conditions. Probes with one or more mismatched bases in the target-specific portion, by contrast, will hybridize to their respective target region to a measurably lesser extent. Both the first probe and the second probe must be hybridized to the target for a ligation product to be generated.

In certain embodiments, highly related sequences that differ by as little as a single nucleotide can be distinguished. For example, according to certain embodiments, one can distinguish the two potential alleles in a biallelic locus as follows. One can combine a ligation probe set comprising two first probes, differing in their primer-specific portions and their pivotal complement (see, e.g., probes A and B in FIG. 3(A)), one second probe (see, e.g., probe Z in FIG. 3(A)), and the sample containing the target. All three probes will hybridize with the target sequence under appropriate conditions (see, e.g., FIG. 3(B)). Only the first probe with the hybridized pivotal complement, however, will be ligated with the hybridized second probe (see, e.g., FIG. 3(C)). Thus, if only one allele is present in the sample, only one ligation product for that target will be generated (see, e.g., ligation product A-Z in FIG. 3(D)). Both ligation products would be formed in a sample from a heterozygous individual. In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide.

In certain embodiments, there may be more than two alleles for a given locus. For example, in certain embodiments, a locus may have one of three or four possible different nucleotides. In certain such embodiments, one may employ three or four different first or second ligation probes that each have a different pivotal complement. In certain embodiments, each of the different probes also has a different primer-specific portion.

Many different double-stranded-dependent labels may be used in various embodiments of the present invention. For example, double-stranded-dependent labels include, but are not limited to, intercalating agents, including, but not limited to, SYBR Green 1, Ethidium Bromide, Acridine Orange, and Hoechst 33258 (all available from Molecular Probes Inc., Eugene, Oreg.); TOTAB, TOED1 and TOED2 (Benson et al., Nucleic Acid Research, 21(24):5727-5735 (1993)); TOTO and YOYO (Benson et al., Analytical Biochemistry, 231:247-255 (1995). Exemplary double-stranded-dependent labels include, but are not limited to, certain minor groove binder dyes, including, but not limited to, 4′,6-diamino-2-phenylindole (Molecular Probes Inc., Eugene, Oregon). Certain of the above-noted double-stranded-dependent labels and others are discussed, e.g., in Handbook of Fluorescent Probes and Research Chemicals, Sixth Edition, by Richard Haugland, Molecular Probes, Inc., Eugene, Oregon (1996) (See, e.g., pages 149 to 151. Certain exemplary double-stranded-dependent labels are described, for example, in U.S. Pat. Nos. 5,994,056 and 6,171,785.

In certain embodiments, one may use a double-stranded-dependent label and a threshold difference between first and second detectable signal values to detect the presence or absence of a target nucleic acid in a sample. In such embodiments, if the difference between the first and second detectable signal values is the same as or greater than the threshold difference, i.e., there is a threshold difference, one concludes that the target nucleic acid is present. If the difference between the first and second detectable signal values is less than the threshold difference, i.e., there is no threshold difference, one concludes that the target nucleic acid is absent.

Certain nonlimiting examples of how one may set a threshold difference according to certain embodiments follow.

First, in certain embodiments, a double-stranded-dependent label that is not exposed to double-stranded nucleic acid may have a first detectable signal value of zero. In certain embodiments, when one carries out an amplification reaction using a composition comprising a double-stranded-dependent label, unligated ligation probes, and appropriate primers for those probes, and known not to contain ligation products, the detectable signal value may increase linearly during and/or after an amplification reaction. (In other words, the second detectable signal value is linearly increased from the first detectable signal value.) In certain such embodiments, when an amplification reaction is carried out with a composition that includes a ligation product and appropriate primers for amplifying the ligation product, the detectable signal value may increase exponentially during and/or after an amplification reaction. (In other words, the second detectable signal value is exponentially increased from the first detectable signal value.)

Thus, in certain such embodiments, one may measure detectable signal values at two or more points during amplification, and at the end of the amplification reaction, to determine if the increase in detectable signal value is linear or exponential. In certain embodiments, one may measure detectable signal values at three or more points during amplification to determine if the increase in detectable signal value is linear or exponential. In certain embodiments, if the increase is exponential, there is a threshold difference between the first and second detectable signal values.

In certain embodiments, one employs threshold time values (Tₜ) to determine whether a particular target nucleic acid sequence is present. In certain such embodiments, the threshold time value is the minimum time of an amplification reaction that results in a set detectable signal value of a label. For example, in certain embodiments, when one carries out an amplification reaction using a composition which comprises a double-stranded-dependent label, unligated ligation probes, and appropriate primers for those probes, and which is known not to contain ligation products, the time that results in a set intensity value 1 may be X seconds. The threshold time value for such a reaction is thus X. In certain such embodiments, when an amplification reaction is carried out with a composition that includes a ligation product and appropriate primers for amplifying the ligation product, the time threshold value may be Y seconds. Thus, the time threshold value for such a reaction is Y.

In certain embodiments, one may use the difference between such threshold time values (ΔTₜ) (here X−Y) to assess whether the target nucleic acid sequence is present. For example, in certain embodiments, one may conclude that a ΔTₜ of somewhere above or equal to a set value slightly above 0 indicates the presence of the target nucleic acid sequence, and value below that threshold indicates the absence of the target nucleic acid sequence. In certain embodiments, one may use the standard deviation of the threshold time value for the amplification reaction without any ligation product to set the appropriate ΔTₜ to signify presence of target nucleic acid sequence. For example, in certain embodiments, if the standard deviation is 1, one can set the minimum ΔTₜ at greater than 1 to signify the presence of target nucleic acid sequence. In certain embodiments, if the standard deviation is 1, one can set the minimum ΔTₜ at greater than 2 to signify the presence of target nucleic acid sequence.

In certain embodiments, one may seek to detect the presence or absence of two different alleles at a particular locus. In certain embodiments, one may use threshold time values to determine if a sample is homozygous for one or the other allele or if the sample is heterozygous containing both alleles. For example, in certain embodiments, one may use two different primer sets in separate amplification reactions for detecting two different alleles. In certain such embodiments one primer set includes primers PA and PZ and another primer set includes primers PB and PZ for detecting alleles A and B, respectively. In certain such embodiments, one may determine the ΔTₜ as follows:

ΔTₜ, =Tₜ. (amplification with primers PB and PZ) minus Tₜ (amplification with primers PA and PZ).

In certain embodiments, one can then set various ΔTₜ values to determine whether the sample is heterozygous or is homozygous for one of the two alleles. For example, in certain embodiments in which Tₜ is in seconds, one may conclude that the sample: is homozygous for allele A if the ΔTₜ is greater than or equal to 270; homozygous for allele B if the ΔTₜ is less than or equal to −120; heterozygous if ΔTₜ is greater than or equal to −60 and less than or equal to 210; and make no call if ΔTₜ is greater than −120 and less than −60 or greater than 210 and less than 270. Also, in certain embodiments, one may conclude that there are no ligation products if the Tₜ of both amplification reactions is greater than the average Tₜ of a control (containing no DNA) minus two standard deviations. In various embodiments, one may set the ranges of ΔTₜ values at other levels as appropriate for determining the presence or absence of various alleles.

In certain embodiments, one employs threshold cycle (Cₜ) values to determine whether a particular target nucleic acid sequence is present. In certain such embodiments, the Cₜ value is the minimum number of cycles in an amplification reaction that result in a set detectable signal value of a label. For example, in certain embodiments, when one carries out an amplification reaction using a composition which comprises a double-stranded-dependent label, unligated ligation probes, and appropriate primers for those probes, and which is known not to contain ligation products, the number of cycles that result in a set intensity value 1 may be 40. The Cₜ value for such a reaction is thus 40. In certain such embodiments, when an amplification reaction is carried out with a composition that includes a ligation product and appropriate primers for amplifying the ligation product, the Cₜ value may be 30. Thus, the Cₜ value for such a reaction is 30.

In certain embodiments, one may use the difference between such Cₜ values (ΔCₜ) (here 40 minus 30=10) to assess whether the target nucleic acid sequence is present. For example, in certain embodiments, one may conclude that a ΔCₜ of somewhere above or equal to a set value slightly above 0 indicates the presence of the target nucleic acid sequence, and value below that threshold indicates the absence of the target nucleic acid sequence. In certain embodiments, one may use the standard deviation of the Cₜ value for the amplification reaction without any ligation product to set the appropriate ΔCₜ to signify presence of target nucleic acid sequence. For example, in certain embodiments, if the standard deviation is 1, one can set the minimum ΔCₜ at greater than 1 to signify the presence of target nucleic acid sequence. In certain embodiments, if the standard deviation is 1, one can set the minimum ΔCₜ at greater than 2 to signify the presence of target nucleic acid sequence.

In certain embodiments, one may seek to detect the presence or absence of two different alleles at a particular locus. In certain embodiments, one may use Cₜ values to determine if a sample is homozygous for one or the other allele or if the sample is heterozygous containing both alleles. For example, in certain embodiments, one may use two different primer sets in separate amplification reactions for detecting two different alleles. In certain such embodiments one primer set includes primers PA and PZ and another primer set includes primers PB and PZ for detecting alleles A and B, respectively. In certain such embodiments, one may determine the ΔCₜ as follows:

ΔCₜ =Cₜ (amplification with primers PB and PZ) minus Cₜ (amplification with primers PA and PZ).

In certain embodiments, one can then set various ΔCₜ values to determine whether the sample is heterozygous or is homozygous for one of the two alleles. For example, in certain embodiments, one may conclude that the sample: is homozygous for allele A if the ΔCₜ is greater than or equal to 4.5; homozygous for allele B if the ΔCₜ is less than or equal to −2; heterozygous if ΔCₜ is greater than or equal to −1 and less than or equal to 3.5; and make no call if ΔCₜ is greater than −2 and less than −1 or greater than 3.5 and less than 4.5. Also, in certain embodiments, one may conclude that there are no ligation products if the Cₜ of both amplification reactions is greater than the average Cₜ of a control (containing no DNA) minus two standard deviations. In various embodiments, one may set the ranges of ΔCₜ values at other levels as appropriate for determining the presence or absence of various alleles.

In certain embodiments one may use Tₜ and/or Cₜ values with various methods employing double-stranded-dependent labels as discussed herein. In certain embodiments, one may use Tₜ, and/or Cₜ values with different types of ligation and amplification methods. For example, one may use Tₜ and/or Cₜ values in any of a variety of methods employing ligation and amplification reactions. Exemplary methods include, but are not limited to, those discussed in U.S. Pat. No. 6,027,889, PCT Published Patent Application No. WO 01/92579, and U.S. patent application Ser. Nos. 09/584,905, 10/011,993, and 60/412,225.

In certain embodiments, one may employ a ligation probe set that can be used in a FEN-OLA technique (FEN is flap endonuclease and OLA is oligonucleotide ligation). In a FEN-OLA technique, a first probe of a ligation probe set comprises a target-specific portion that is designed to hybridize to the target nucleic acid sequence. A second probe of the ligation probe set comprises a flap portion, a target-specific portion, and a FEN cleavage position nucleotide between the flap portion and the target-specific portion. The target-specific portion of the second probe is designed to hybridize to the target nucleic acid sequence such the end of the target-specific portion nearest the flap portion is adjacent to the hybridized target-specific portion of the first probe.

The flap portion is designed such that a substantial portion of the flap portions do not hybridize to the target nucleic acid sequence. A “substantial portion of the flap portions do not hybridize” refers to a portion of the total number of flap portions, and it does not refer to a portion of an individual flap portion. In certain embodiments, “a substantial portion of flap portions that do not hybridize” means that at least 90% of the flap portions do not hybridize. In certain embodiments, at least 95% of the flap portions do not hybridize.

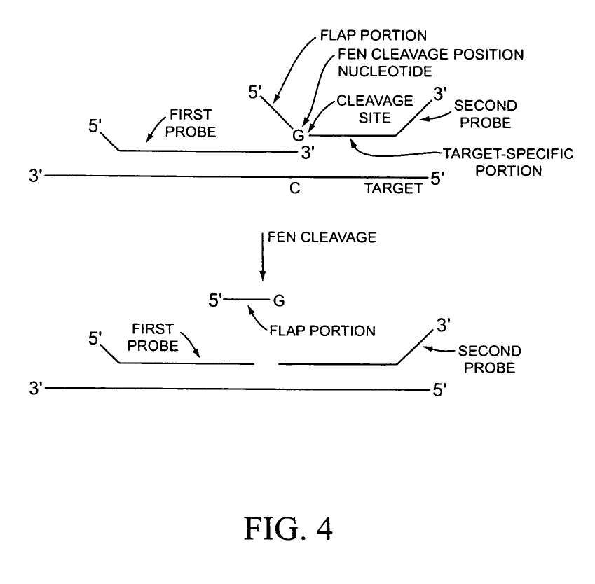

FEN will cleave the second probe between the cleavage position nucleotide and the target-specific portion, if the proper target nucleic acid sequence is present. Specifically, such cleavage occurs it the target-specific portions of the first and second probes hybridize to the target nucleic acid sequence, and the FEN cleavage position nucleotide is complementary to the nucleotide of the target nucleic acid sequence that is directly adjacent to the portion of the target nucleic sequence that hybridizes to the target specific portion of the second probe. FIG. 4 shows certain nonlimiting examples that help to illustrate certain ligation probe sets that may be used in FEN-OLA techniques according to certain embodiments.

If the flap is cleaved, the second probe may then be ligated to the adjacent hybridized first probe of a ligation probe set. If the flap is not cleaved, the second probe will not be ligated to the adjacent hybridized first probe.

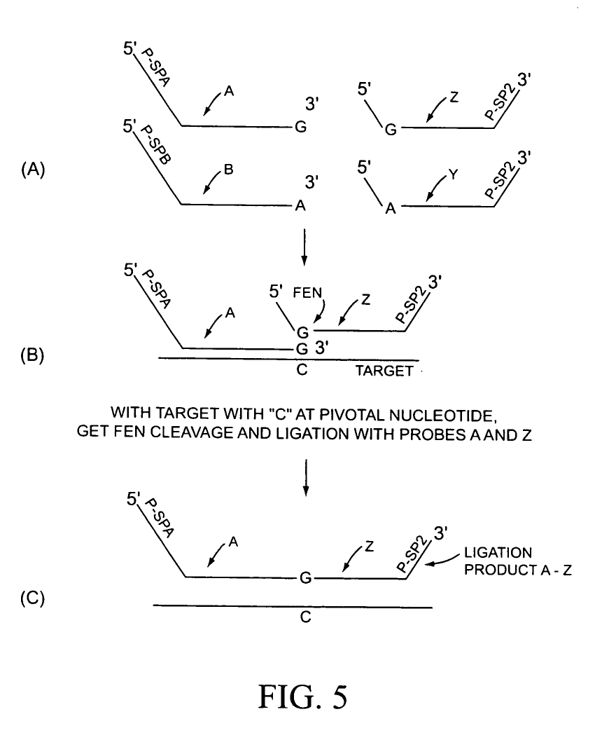

Certain nonlimiting examples of probes used in a FEN-OLA technique are depicted in FIG. 5. In FIG. 5, one employs a probe set comprising: two first probes, differing in their primer-specific portions and their pivotal complements (see, e.g., probes A and B in FIG. 5(A)); and two second probes that comprise different FEN cleavage position nucleotides that correspond to the pivotal complements of the two first probes (see, e.g., probes Y and Z in FIG. 5(A)).

In the embodiment shown in FIG. 5, FEN will cleave the flap of a second probe only if the second probe comprises a FEN cleavage position nucleotide that is complementary to the pivotal nucleotide of target nucleic acid sequence (see, e.g., FIG. 5(B)). In such a situation in such embodiments, the first and second probes of the probe set are ligated together if the pivotal complement of the first probe is complementary to the pivotal nucleotide of the target nucleic acid sequence (see, e.g., FIG. 5(C)). If there is a mismatch at the pivotal nucleotide, no ligation occurs.

Thus, if only one allele is present in the sample, only one ligation product for that target will be generated (see, e.g., ligation product A-Z in FIG. 5(C)). Both ligation products would be formed in a sample from a heterozygous individual. In certain embodiments, cleavage of probes with a FEN cleavage position nucleotide that is not complementary to the pivotal nucleotide may occur, but such cleavage occurs to a measurably lesser extent than cleavage of probes with a FEN cleavage position nucleotide that is complementary to the pivotal nucleotide. In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide.

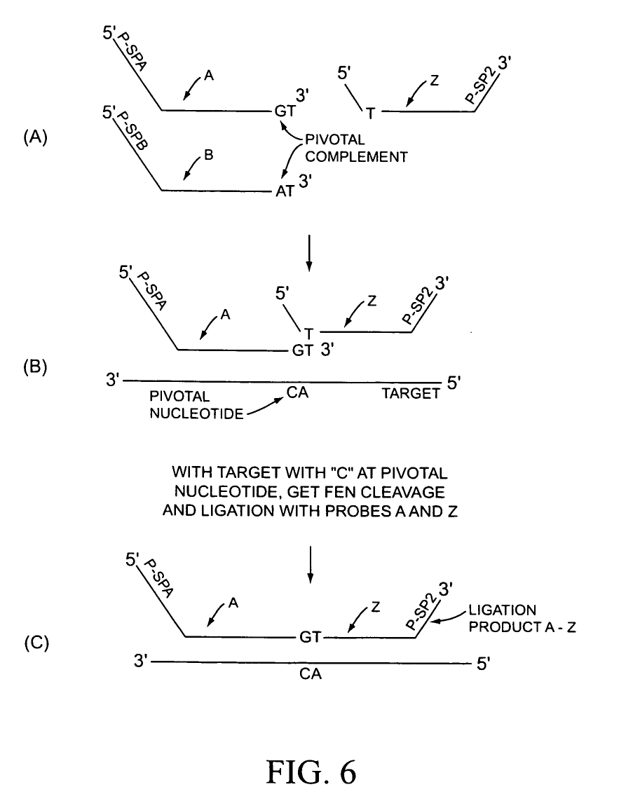

Certain nonlimiting examples of probes used in a FEN-OLA technique are also depicted in FIG. 6. In FIG. 6, one employs a probe set comprising two first probes, which comprise different primer-specific portions and different pivotal complements and the pivotal complement of each first probe is at the penultimate nucleotide position at the 3′ end of the first probes (see, e.g., probes A and B in FIG. 6(A)). The probe set further comprises a second probe that comprises a FEN cleavage position nucleotide that is the same as the nucleotide at the 3′ end of the two first probes (see, e.g., probe Z in FIG. 6(A)).

In the embodiment depicted in FIG. 6, FEN will cleave the flap of a second probe only if the second probe comprises a FEN cleavage position nucleotide that is complementary to the nucleotide immediately 5′ of the pivotal nucleotide of target nucleic acid sequence (see, e.g., FIG. 6(B)). In such a situation in such embodiments, the first and second probes of the probe set are ligated together if: (1) the pivotal complement of the first probe is complementary to the pivotal nucleotide of the target nucleic acid sequence; and (2) the nucleotide at the 3′ end of the first probe is complementary to the nucleotide immediately 5′ of the pivotal nucleotide of target nucleic acid sequence (see, e.g., FIG. 6(C)). If there is a mismatch at the pivotal nucleotide, no ligation occurs.

Thus, if only one allele is present in the sample, only one ligation product for that target will be generated (see, e.g., ligation product A-Z in FIG. 6(C)). Both ligation products would be formed in a sample from a heterozygous individual. In certain embodiments, cleavage of probes with a FEN cleavage position nucleotide that is not complementary to the nucleotide immediately 5′ of the pivotal nucleotide may occur, but such cleavage occurs to a measurably lesser extent than cleavage of probes with a FEN cleavage position nucleotide that is complementary to the nucleotide immediately 5′ of the pivotal nucleotide. In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide. In certain embodiments, ligation of first probes with a nucleotide at the 3′ end that is not complementary to the nucleotide immediately 5′ of the pivotal nucleotide may occur, but such ligation occurs to a measurably lesser extent than ligation of first probes with a nucleotide at the 3′ end that is complementary to the nucleotide immediately 5′ of the pivotal nucleotide.

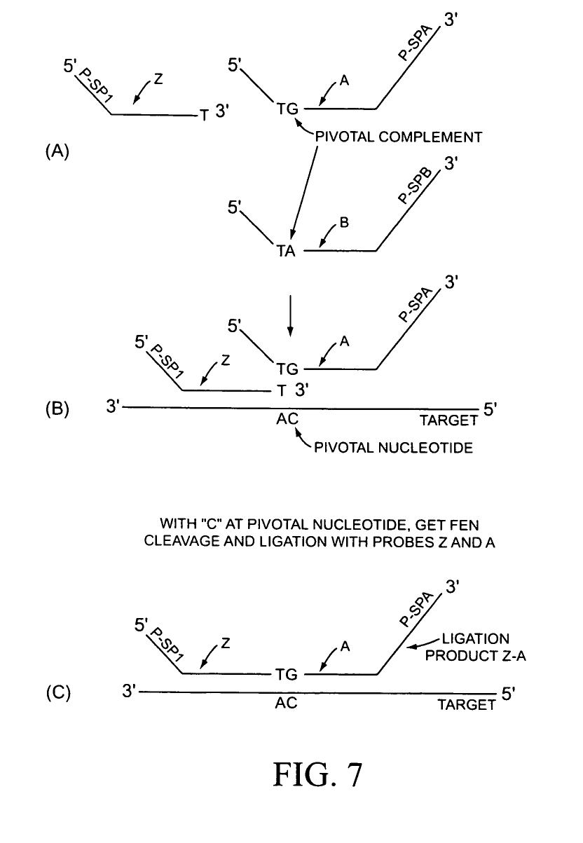

Certain nonlimiting examples of probes used in a FEN-OLA technique are also depicted in FIG. 7. In FIG. 7, one employs a probe set comprising two second probes, which comprise the same FEN cleavage position nucleotide and comprise different primer-specific portions and different pivotal complements (the pivotal complement of each second probe is immediately 3′ to the FEN cleavage position nucleotide) (see, e.g., probes A and B in FIG. 7(A)). The probe set further comprises a first probe that comprises a nucleotide at the 3′ end that is the same as the FEN cleavage position nucleotide (see, e.g., probe Z in FIG. 7(A)).

In the embodiment depicted in FIG. 7, FEN will cleave the flap of a second probe only if the second probe comprises a FEN cleavage position nucleotide that is complementary to the nucleotide immediately 3′ of the pivotal nucleotide of target nucleic acid sequence (see, e.g., FIG. 7(B)). In such a situation in such embodiments, the first and second probes of the probe set are ligated together if: (1) the pivotal complement of the second probe is complementary to the pivotal nucleotide of the target nucleic acid sequence; and (2) the nucleotide at the 3′ end of the first probe is complementary to the nucleotide immediately 3′ of the pivotal nucleotide of target nucleic acid sequence (see, e.g., FIG. 7(C)). If there is a mismatch at the pivotal nucleotide, no ligation occurs.

Thus, if only one allele is present in the sample, only one ligation product for that target will be generated (see, e.g., ligation product Z-A in FIG. 7(C)). Both ligation products would be formed in a sample from a heterozygous individual. In certain embodiments, cleavage of probes with a FEN cleavage position nucleotide that is not complementary to the nucleotide immediately 3′ of the pivotal nucleotide may occur, but such cleavage occurs to a measurably lesser extent than cleavage of probes with a FEN cleavage position nucleotide that is complementary to the nucleotide immediately 3′ of the pivotal nucleotide. In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide. In certain embodiments, ligation of first probes with a nucleotide at the 3′ end that is not complementary to the nucleotide immediately 3′ of the pivotal nucleotide may occur, but such ligation occurs to a measurably lesser extent than ligation of first probes with a nucleotide at the 3′ end that is complementary to the nucleotide immediately 3′ of the pivotal nucleotide.

In certain embodiments, one may increase the length of the probes by including sequences that have a specific portion that is designed to hybridize to a particular target nucleic acid sequence and an adjacent degenerate portion. For example, in certain embodiments, a group of probes may all be used for a specific six nucleotide portion of a particular target nucleic acid sequence. In certain such embodiments, each of the probes in the group may comprise the same six nucleotide sequence portion that is complementary to the particular target nucleic acid sequence. The probes in the group further comprise additional adjacent degenerate portions that randomly have the four different nucleotides at each of the positions of the degenerate portion so that both the specific six nucleotide portion and the degenerate portion of at least one of the probes in the group will hybridize to any nucleic acid that includes the specific six nucleotide portion.

For example, for a given six nucleotide target nucleic acid sequence, each probe of a group of probes may include the same six nucleotide sequence portion that is complementary to the particular target nucleic acid sequence. Each of the probes of the group may further comprise a four nucleotide degenerate portion. The probes in the series may have all of the possible combinations for a four nucleotide sequence. Thus, although only six nucleotides provide specificity for the target nucleic acid sequence, one of the probes in the group will have a random four nucleotide sequence that will also hybridize to the target. Accordingly, the length of the portion of at least one probe in the group that hybridizes to the target increases to ten nucleotides rather than six nucleotides.

In certain embodiments, one may increase the length of the probe by adding a portion with universal nucleotides that will hybridize to most or all nucleotides nonspecifically. Exemplary, but nonlimiting, universal nucleotides are discussed, e.g., in Berger et al. Angew. Chem. Int. Ed. Engl. (2000) 39:2940-42; and Smith et al. Nucleosides & Nucleotides (1998) 17: 541-554. An exemplary, but nonlimiting, universal nucleotide is 8-aza-7-deazaadenine, which is discussed, e.g., in Sella and Debelak, Nucl. Acids Res., 28:3224-3232 (2000).

In certain embodiments, one may employ universal nucleotides or degenerate portions in probes to accommodate sequence variation.

A primer set according to certain embodiments comprises at least one primer capable of hybridizing with the primer-specific portion of at least one probe of a ligation probe set. In certain embodiments, a primer set comprises at least one first primer and at least one second primer, wherein the at least one first primer specifically hybridizes with one probe of a ligation probe set (or a complement of such a probe) and the at least one second primer of the primer set specifically hybridizes with a second probe of the same ligation probe set (or a complement of such a probe). In certain embodiments, the first and second primers of a primer set have different hybridization temperatures, to permit temperature-based asymmetric PCR reactions.

The skilled artisan will appreciate that while the probes and primers of the invention may be described in the singular form, a plurality of probes or primers may be encompassed by the singular term, as will be apparent from the context. Thus, for example, in certain embodiments, a ligation probe set typically comprises a plurality of first probes and a plurality of second probes.

The criteria for designing sequence-specific primers and probes are well known to persons of ordinary skill in the art. Detailed descriptions of primer design that provide for sequence-specific annealing can be found, among other places, in Diffenbach and Dveksler, PCR Primer, A Laboratory Manual, Cold Spring Harbor Press, 1995, and Kwok et al. (Nucl. Acid Res. 18:999-1005, 1990). The sequence-specific portions of the primers are of sufficient length to permit specific annealing to complementary sequences in ligation products and amplification products, as appropriate.

According to certain embodiments, a primer set of the present invention comprises at least one second primer. In certain embodiments, the second primer in that primer set is designed to hybridize with a 3′ primer-specific portion of a ligation or amplification product in a sequence-specific manner (see, e.g., FIG. 2C). In certain embodiments, the primer set further comprises at least one first primer. In certain embodiments, the first primer of a primer set is designed to hybridize with the complement of the 5′ primer-specific portion of that same ligation product or amplification product in a sequence-specific manner.

A universal primer or primer set may be employed according to certain embodiments. In certain embodiments, a universal primer or a universal primer set hybridizes with two or more of the probes, ligation products, or amplification products in a reaction, as appropriate. When universal primer sets are used in certain amplification reactions, such as, but not limited to, PCR, qualitative or quantitative results may be obtained for a broad range of template concentrations.

In certain embodiments involving a ligation reaction and an amplification reaction, one may employ at least one probe and/or at least one primer that includes a minor groove binder attached to it. Certain exemplary minor groove binders and certain exemplary methods of attaching minor groove binders to oligonucleotides are discussed, e.g., in U.S. Pat. Nos. 5,801,155 and 6,084,102. Certain exemplary minor groove binders are those available from Epoch Biosciences, Bothell, Washington. According to certain embodiments, a minor groove binder may be attached to at least one of the following: at least one probe of a ligation probe set and at least one primer of a primer set.

According to certain embodiments, a minor groove binder is attached to a ligation probe that includes a 3′ primer-specific portion. In certain such embodiments, the presence of the minor groove binder facilitates use of a short primer that hybridizes to the 3′ primer-specific portion in an amplification reaction. For example, in certain embodiments, the short primer, or segment of the primer that hybridizes to the primer-specific portion or its complement, may have a length of anywhere between 8 and 15 nucleotides.

In certain embodiments, a minor groove binder is attached to at least one of a forward primer and a reverse primer to be used in an amplification reaction. In certain such embodiments, a primer with a minor groove binder attached to it may be a short primer. For example, in certain embodiments, the short primer, or segment of the primer that hybridizes to the primer-specific portion or its complement, may have a length of anywhere between 8 and 15 nucleotides. In certain embodiments, both the forward and reverse primers may have minor groove binders attached to them.

In certain embodiments, one may use minor groove binders as follows in methods that employ a ligation probe set comprising: a first probe comprising a 5′ primer-specific portion; and a second probe comprising a 3′ primer-specific portion. A minor groove binder is attached to the 3′ end of the second probe, and a minor groove binder is attached to a primer that hybridizes to the complement of the 5′ primer-specific portion of the first probe. In certain such embodiments, the presence of the minor groove binders facilitates use of short forward and reverse primers in an amplification reaction. For example, in certain embodiments, the short primer, or segment of the primer that hybridizes to the primer-specific portion or its complement, may have a length of anywhere between 8 and 15 nucleotides.

In certain embodiments, one may employ non-natural nucleotides other than the naturally occurring nucleotides A, G, C, T, and U. For example, in certain embodiments, one may employ primer-specific portions and primers that comprise pairs of non-natural nucleotides that specifically hybridize to one another and not to naturally occurring nucleotides. Exemplary, but nonlimiting, non-natural nucleotides are discussed, e.g., in Wu et al. J. Am. Chem. Soc. (2000) 122: 7621-32; Berger et al. Nuc. Acids Res. (2000) 28: 2911-14, Ogawa et al. J. Am. Chem. Soc. (2000) 122: 3274-87

Certain embodiments include a ligation agent. For example, ligase is an enzymatic ligation agent that, under appropriate conditions, forms phosphodiester bonds between the 3′-OH and the 5′-phosphate of adjacent nucleotides in DNA or RNA molecules, or hybrids. Exemplary ligases include, but are not limited to, Tth K294R ligase and Tsp AK16D ligase. See, e.g., Luo et al., Nucleic Acids Res., 24(14):3071-3078 (1996); Tong et al., Nucleic Acids Res., 27(3):788-794 (1999); and Published PCT Application No. WO 00/26381. Temperature sensitive ligases, include, but are not limited to, T4 DNA ligase, T7 DNA ligase, and E. coli ligase. In certain embodiments, thermostable ligases include, but are not limited to, Taq ligase, Tth ligase, Tsc ligase, and Pfu ligase. Certain thermostable ligases may be obtained from thermophilic or hyperthermophilic organisms, including but not limited to, prokaryotic, eukaryotic, or archael organisms. Certain RNA ligases may be employed in certain embodiments. In certain embodiments, the ligase is a RNA dependent DNA ligase, which may be employed with RNA template and DNA ligation probes. An exemplary, but nonlimiting example, of a ligase with such RNA dependent DNA ligase activity is T4 DNA ligase. In certain embodiments, the ligation agent is an “activating” or reducing agent.

Chemical ligation agents include, without limitation, activating, condensing, and reducing agents, such as carbodiimide, cyanogen bromide (BrC N), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of certain embodiments of the invention. Detailed protocols for chemical ligation methods and descriptions of appropriate reactive groups can be found, among other places, in Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

In certain embodiments, at least one polymerase is included. In certain embodiments, at least one thermostable polymerase is included. Exemplary thermostable polymerases, include, but are not limited to, Taq polymerase, Pfx polymerase, Pfu polymerase, Vent® polymerase, Deep Vent™ polymerase, Pwo polymerase, Tth polymerase, UITma polymerase and enzymatically active mutants and variants thereof. Descriptions of these polymerases may be found, among other places, at the world wide web URL: the-scientist.com/yr1998/jan/profile 1_{—}980105. html; at the world wide web URL: the-scientist.com/yr2001/jan/profile_{—}010903. html; at the world wide web URL: the-scientist.com/yr2001/sep/profile2 ₁₃010903. html; at the article The Scientist 12(1):17 (Jan. 5, 1998); and at the article The Scientist 15(17):1 (Sep. 3, 2001).

The skilled artisan will appreciate that the complement of the disclosed probe, target, and primer sequences, or combinations thereof, may be employed in certain embodiments of the invention. For example, without limitation, a genomic DNA sample may comprise both the target sequence and its complement. Thus, in certain embodiments, when a genomic sample is denatured, both the target sequence and its complement are present in the sample as single-stranded sequences. In certain embodiments, ligation probes may be designed to specifically hybridize to an appropriate sequence, either the target sequence and/or its complement.

C. Certain Exemplary Component Methods

Ligation according to the present invention comprises any enzymatic or chemical process wherein an internucleotide linkage is formed between the opposing ends of nucleic acid sequences that are adjacently hybridized to a template. Additionally, the opposing ends of the annealed nucleic acid sequences should be suitable for ligation (suitability for ligation is a function of the ligation method employed). The internucleotide linkage may include, but is not limited to, phosphodiester bond formation. Such bond formation may include, without limitation, those created enzymatically by a DNA or RNA ligase, such as bacteriophage T4 DNA ligase, T4 RNA ligase, T7 DNA ligase, Thermus thermophilus (Tth) ligase, Thermus aquaticus (Taq) ligase, or Pyrococcus furiosus (Pfu) ligase. Other internucleotide linkages include, without limitation, covalent bond formation between appropriate reactive groups such as between an α-haloacyl group and a phosphothioate group to form a thiophosphorylacetylamino group; and between a phosphorothioate and a tosylate or iodide group to form a 5′-phosphorothioester or pyrophosphate linkages.

In certain embodiments, chemical ligation may, under appropriate conditions, occur spontaneously such as by autoligation. Alternatively, in certain embodiments, “activating” or reducing agents may be used. Examples of activating agents and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light. Non-enzymatic ligation according to certain embodiments may utilize specific reactive groups on the respective 3′ and 5′ ends of the aligned probes.

In certain embodiments, ligation generally comprises at least one cycle of ligation, for example, the sequential procedures of: hybridizing the target-specific portions of a first probe and a second probe, that are suitable for ligation, to their respective complementary regions on a target nucleic acid sequence; ligating the 3′ end of the first probe with the 5′ end of the second probe to form a ligation product; and denaturing the nucleic acid duplex to separate the ligation product from the target nucleic acid sequence. The cycle may or may not be repeated. For example, without limitation in certain embodiments, thermocycling the ligation reaction may be employed to linearly increase the amount of ligation product.

According to certain embodiments, one may use ligation techniques such as gap-filling ligation, including, without limitation, gap-filling OLA and LCR, bridging oligonucleotide ligation, FEN-LCR, and correction ligation. Descriptions of these techniques can be found, among other places, in U.S. Pat. No. 5,185,243, published European Patent Applications EP 320308 published PCT Patent Application WO 90/01069, published PCT Patent Application WO 02/02823, and U.S. patent application Ser. No. 09/898,323.

In certain embodiments, one may employ poly-deoxy-inosinic-deoxy-cytidylic acid (Poly [d(I-C)]) (Available in Roche Applied Science catalog, 2002) in a ligation reaction. In certain embodiments, one uses any number between 15 to 80 ng/microliter of Poly [d(I-C)] in a ligation reaction. In certain In certain embodiments, one uses 30 ng/microliter of Poly [d(I-C)] in a ligation reaction.

One may use Poly ld(I-C)) in a ligation reaction with various methods employing ligation probes discussed herein. In certain embodiments, one may use Poly [d(I-C)] with different types of ligation methods. For example, one may use Poly [d(I-C)] in any of a variety of methods employing ligation reactions. Exemplary methods include, but are not limited to, those discussed in U.S. Pat. No. 6,027,889, PCT Published Patent Application No. WO 01/92579, and U.S. patent application Ser. Nos. 09/584,905; 10/011,993; and 60/412,225.

In certain embodiments, in a ligation reaction, one may employ unrelated double-stranded nucleic acid that does not include a sequence that is the same as or is similar to the target nucleic acid sequence that is sought. In certain such embodiments, such double-stranded nucleic acid also will not include a sequence that is the same as or is similar to the sequences of the primer-specific portions of the ligation probes. In certain such embodiments, such double-stranded nucleic acid also will not include a sequence that is the same as or is similar to the sequences of the target-specific portions of the ligation probes. In certain embodiments, one may employ double-stranded poly A and poly T nucleic acid. In certain embodiments, one may employ double-stranded poly G and poly C nucleic acid. In certain such embodiments, one may employ nucleic acid from an organism unrelated to the organism from which the target nucleic acid sequence is derived. In certain embodiments, one may employ bacterial nucleic acid. In certain embodiments, one may employ viral DNA. In certain embodiments, one may employ plasmid DNA. In certain embodiments, the double-stranded nucleic acid assists in reducing the amount of ligation that may occur between ligation probes when the sought target nucleic acid sequence is not present.

In certain embodiments, one uses any number between 15 to 80 ng/microliter of unrelated double-stranded nucleic acid in a ligation reaction. In certain embodiments, one uses 30 ng/microliter of unrelated double-stranded nucleic acid in a ligation reaction.

One may use unrelated double-stranded nucleic acid in a ligation reaction with various methods employing ligation probes discussed herein. In certain embodiments, one may use unrelated double-stranded nucleic acid with different types of ligation methods. For example, one may use unrelated double-stranded nucleic acid in any of a variety of methods employing ligation reactions. Exemplary methods include, but are not limited to, those discussed in U.S. Pat. No. 6,027,889, PCT Published Patent Application No. WO 01/92579, and U.S. patent application Ser. Nos. 09/584,905; 10/011,993; and 60/412,225.

Exemplary, but nonlimiting ligation reaction conditions may be as follows. In certain embodiments, the ligation reaction temperature may range anywhere from about 45° C. to 55° C. for anywhere from two to 10 minutes. In certain embodiments, any number from 2 to 100 cycles of ligation are performed. In certain embodiments, 60 cycles of ligation are performed. In certain embodiments, allele specific ligation probes (a probe of a probe set that is specific to a particular allele at a given locus) are in a concentration anywhere from 2 to 100 nM. In certain embodiments, allele specific ligation probes are in a concentration of 50 nM. In certain embodiments, allele specific ligation probes are in a concentration anywhere from 1 to 7 nM. In certain embodiments, the locus specific ligation probes (a probe of a probe set that is not specific to a particular allele, but is specific for a given locus) are in a concentration anywhere from 2 to 200 nM. In certain embodiments, locus specific ligation probes are in a concentration of 100 nM. In certain embodiments, fragmented genomic DNA is in a concentration anywhere from 5 ng/μl to 200 ng/μl in the ligation reaction. In certain embodiments, fragmented genomic DNA is in a concentration of 130 ng/μl in the ligation reaction. In certain embodiments, the pH for the ligation reaction is anywhere from 7 to 8. In certain embodiments, the Mg++ concentration is anywhere from 2 to 22 nM. In certain embodiments, the ligase concentration is anywhere from 0.04 to 0.16 u/μl. In certain embodiments, the ligase concentration is anywhere from 0.02 to 0.12 u/μl. In certain embodiments, the K+ concentration is anywhere from 0 to 70 mM. In certain embodiments, the K+ concentration is anywhere from 0 to 20 mM. In certain embodiments, the Poly [d(I-C)] concentration is anywhere from 0 to 30 ng/μl. In certain embodiments, the Poly [d(I-C)] concentration is anywhere from 0 to 20 ng/μl. In certain embodiments, the NAD+ concentration is anywhere from 0.25 to 2.25 mM.

In certain embodiments, one forms a test composition for a subsequent amplification reaction by subjecting a ligation reaction composition to at least one cycle of ligation. In certain embodiments, after ligation, the test composition may be used directly in the subsequent amplification reaction. In certain embodiments, prior to the amplification reaction, the test composition may be subjected to a purification technique that results in a test composition that includes less than all of the components that may have been present after the at least one cycle of ligation. For example, in certain embodiments, one may purify the ligation product.

Purifying the ligation product according to certain embodiments comprises any process that removes at least some unligated probes, target nucleic acid sequences, enzymes, and/or accessory agents from the ligation reaction composition following at least one cycle of ligation. Such processes include, but are not limited to, molecular weight/size exclusion processes, e.g., gel filtration chromatography or dialysis, sequence-specific hybridization-based pullout methods, affinity capture techniques, precipitation, adsorption, or other nucleic acid purification techniques. The skilled artisan will appreciate that purifying the ligation product prior to amplification in certain embodiments reduces the quantity of primers needed to amplify the ligation product, thus reducing the cost of detecting a target sequence. Also, in certain embodiments, purifying the ligation product prior to amplification may decrease possible side reactions during amplification and may reduce competition from unligated probes during hybridization.

Hybridization-based pullout (HBP) according to certain embodiments of the present invention comprises a process wherein a nucleotide sequence complementary to at least a portion of one probe (or its complement), for example, the primer-specific portion, is bound or immobilized to a solid or particulate pullout support (see, e.g., U.S. Pat. No. 6,124,092). In certain embodiments, a composition comprising ligation product, target sequences, and unligated probes is exposed to the pullout support. The ligation product, under appropriate conditions, hybridizes with the support-bound sequences. In certain embodiments, the unbound components of the composition are removed, substantially purifying the ligation products from those ligation reaction composition components that do not contain sequences complementary to the sequence on the pullout support. One subsequently removes the purified ligation products from the support and combines them with at least one primer set to form a first amplification reaction composition. The skilled artisan will appreciate that, in certain embodiments, additional cycles of HBP using different complementary sequences on the pullout support may remove all or substantially all of the unligated probes, further purifying the ligation product.

In certain embodiments, one may substantially remove certain unligated probes employing a probe set that includes a binding moiety on either the 5′ end of the first probe or the 3′ end of the second probe. In certain such embodiments, after a ligation reaction, one exposes the composition to a support that binds to the binding moiety. In certain embodiments, the unbound components of the composition are removed, substantially purifying the ligation products from those ligation reaction composition components that do not include the binding moiety, including the unligated probes without a binding moiety. In certain such embodiments, one may then remove the bound components from the support, and then expose them to a support with a bound sequence that is complementary to a portion of the ligation probe without the binding moiety, and that is not complementary to a portion of the ligation probe with the binding moiety. Thus, in certain such embodiments, the unligated first and second probes will be substantially removed from the ligation product. In certain embodiments, one may reverse the process by exposing the composition first to the support with the complementary sequence and second to the support that binds to the binding moiety. In certain embodiments, the binding moiety is biotin, which binds to streptavidin on the support.

In certain embodiments, one may employ different binding moieties (e.g., a first binding moiety and a second binding moiety) on the first probe and second probe of a probe set. In certain such embodiments, after a ligation reaction, one may then expose the composition to a first support that binds one of the binding moieties to capture ligation product and unligated probe with the first binding moiety. In certain embodiments, after removing unbound components, one may then remove the bound components and expose them to a second support that binds the second binding moiety to capture ligation product.

In certain embodiments, one may substantially remove unligated ligation probes using certain exonucleases that act specifically on single stranded nucleic acid. For example, in certain embodiments, one may employ a ligation probe set or sets that include a protective group on one end such that, when the ligation probes are ligated to one another, both ends of the ligation product will be protected from exonuclease digestion. In such embodiments, unligated probes are not protected on one end such that unligated probes are digested by exonuclease. In certain such embodiments, the 5′ end of the first probe includes a protective group, and the 3′ end of the second probe includes a protective group. One skilled in the art will appreciate certain exonucleases and certain protective groups that may be employed according to certain embodiments. In certain embodiments, biotin is used as a protective group. In certain embodiments, one may employ a method such that the exonuclease activity is substantially removed prior to an amplification reaction. In certain embodiments, one may employ an exonuclease that loses activity when exposed to a particular temperature for a given amount of time.

Amplification according to the present invention encompasses a broad range of techniques for amplifying nucleic acid sequences, either linearly or exponentially. Exemplary amplification techniques include, but are not limited to, PCR or any other method employing a primer extension step, and transcription or any other method of generating at least one RNA transcription product. Other nonlimiting examples of amplification are ligase detection reaction (LDR), and ligase chain reaction (LCR). Other nonlimiting examples of amplification are whole-genome amplification reactions. Amplification methods may comprise thermal-cycling or may be performed isothermally. The term “amplification product” includes products from any number of cycles of amplification reactions, primer extension reactions, and RNA transcription reactions, unless otherwise apparent from the context.

In certain embodiments, amplification methods comprise at least one cycle of amplification, for example, but not limited to, the sequential procedures of: hybridizing primers to primer-specific portions of the ligation product or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated. In certain embodiments, amplification methods comprise at least one cycle of amplification, for example, but not limited to, the sequential procedures of: interaction of a polymerase with a promoter; synthesizing a strand of nucleotides in a template-dependent manner using a polymerase; and denaturing the newly-formed nucleic acid duplex to separate the strands. The cycle may or may not be repeated.

Descriptions of certain amplification techniques can be found, among other places, in H. Ehrlich et al., Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000); Sambrook and Russell, Ausbel et al.

Primer extension according to the present invention is an amplification process comprising elongating a primer that is annealed to a template in the 5′ to 3′ direction using a template-dependent polymerase. According to certain embodiments, with appropriate buffers, salts, pH, temperature, and nucleotide triphosphates, including analogs and derivatives thereof, a template dependent polymerase incorporates nucleotides complementary to the template strand starting at the 3′-end of an annealed primer, to generate a complementary strand. Detailed descriptions of primer extension according to certain embodiments can be found, among other places in Sambrook et al., Sambrook and Russell, and Ausbel et al.

Transcription according to certain embodiments is an amplification process comprising an RNA polymerase interacting with a promoter on a single- or double-stranded template and generating a RNA polymer in a 5′ to 3′ direction. In certain embodiments, the transcription reaction composition further comprises transcription factors. RNA polymerases, including but not limited to T3, T7, and SP6 polymerases, according to certain embodiments, can interact with double-stranded promoters. Detailed descriptions of transcription according to certain embodiments can be found, among other places in Sambrook et al., Sambrook and Russell, and Ausbel et al.

Certain embodiments of amplification may employ multiplex amplification, in which multiple target sequences are simultaneously amplified (see, e.g., H. Geada et al., Forensic Sci. Int. 108:31-37 (2000) and D.G. Wang et al., Science 280:1077-82 (1998)).

Methods of optimizing amplification reactions are well known to those skilled in the art. For example, it is well known that PCR may be optimized by altering times and temperatures for annealing, polymerization, and denaturing, as well as changing the buffers, salts, and other reagents in the reaction composition. Optimization may also be affected by the design of the amplification primers used. For example, the length of the primers, as well as the G-C:A-T ratio may alter the efficiency of primer annealing, thus altering the amplification reaction. See James G. Wetmur, “Nucleic Acid Hybrids, Formation and Structure,” in Molecular Biology and Biotechnology, pp.605-8, (Robert A. Meyers ed., 1995).

In certain amplification reactions, one may use dUTP and uracil-N-glycosidase (UNG). Discussion of use of dUTP and UNG may be found, for example, in Kwok et al., “Avoiding false positives with PCR,” Nature, 339:237-238 (1989); and Longo et al. “Use of uracil DNA glycosylase to control carry-over contamination in polymerase chain reactions,” Gene, 93:125-128 (1990).

To detect whether a particular sequence is present, in certain embodiments, a double-stranded-dependent label is included in the amplification reaction. According to certain embodiments, the double-stranded-dependent label indicates the presence or absence (or amount) of a specific nucleic acid sequence in the reaction.

In certain embodiments, the amount of double-stranded-dependent label that gives a signal typically relates to the amount of nucleic acid produced in the amplification reaction. Thus, in certain embodiments, the amount of signal is related to the amount of product created in the amplification reaction. In such embodiments, one can therefore measure the amount of amplification product by measuring the intensity of the signal. According to certain embodiments, one can employ an internal standard to quantify the amplification product indicated by the signal. See, e.g., U.S. Pat. No. 5,736,333.

Devices have been developed that can perform a thermal cycling reaction with compositions containing a fluorescent indicator, emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; and 6,174,670, and include, but are not limited to the ABI Prism® 7700 Sequence Detection System (Applied Biosystems, Foster City, Calif.) and the ABI GeneAmp® 5700 Sequence Detection System (Applied Biosystems, Foster City, Calif.).

In certain embodiments, each of these functions may be performed by separate devices. For example, if one employs a Q-beta replicase reaction for amplification, the reaction may not take place in a thermal cycler, but could include a light beam emitted at a specific wavelength, detection of the fluorescent signal, and calculation and display of the amount of amplification product.

In certain embodiments, combined thermal cycling and fluorescence detecting devices can be used for precise quantification of target nucleic acid sequences in samples. In certain embodiments, fluorescent signals can be detected and displayed during and/or after one or more thermal cycles, thus permitting monitoring of amplification products as the reactions occur in “real time.” In certain embodiments, one can use the amount of amplification product and number of amplification cycles to calculate how much of the target nucleic acid sequence was in the sample prior to amplification.

According to certain embodiments, one could simply monitor the amount of amplification product after a predetermined number of cycles sufficient to indicate the presence of the target nucleic acid sequence in the sample. One skilled in the art can easily determine, for any given sample type, primer sequence, and reaction condition, how many cycles are sufficient to determine the presence of a given target polynucleotide.

According to certain embodiments, the amplification products can be scored as positive or negative as soon as a given number of cycles is complete. In certain embodiments, the results may be transmitted electronically directly to a database and tabulated. Thus, in certain embodiments, large numbers of samples may be processed and analyzed with less time and labor required.

D. Certain Exemplary Embodiments of Detecting Targets

The present invention is directed to methods, reagents, and kits for detecting the presence or absence of (or quantitating) target nucleic acid sequences in a sample, using ligation and amplification reactions. When a particular target nucleic acid sequence is present in a sample, a ligation product is formed that includes at least one particular primer-specific portion. Double-stranded-dependent labels are employed that provide a different detectable signal value depending upon whether a double-stranded nucleic acid is present or absent.

In certain embodiments, one or more nucleic acid species are subjected to ligation and amplification reactions, either directly or via an intermediate, such as a cDNA target generated from an mRNA by reverse transcription or a whole-genome amplification reaction. In certain embodiments, the initial nucleic acid comprises mRNA and a reverse transcription reaction may be performed to generate at least one cDNA, followed by at least one ligation reaction and at least one amplification reaction. In certain embodiments, DNA ligation probes hybridize to target RNA, and an RNA dependent DNA ligase is employed in a ligation reaction, followed by an amplification reaction. The ligation products and amplification products may be detected (or quantitated) using labeled probes.

In certain embodiments, for each target nucleic acid sequence to be detected, a ligation probe set, comprising at least one first probe and at least one second probe, is combined with the sample to form a ligation reaction composition. In certain embodiments, the ligation composition may further comprise a ligation agent. In certain embodiments, the first and second probes in each ligation probe set are suitable for ligation together and are designed to hybridize to adjacent sequences that are present in the target nucleic acid sequence. When the target nucleic acid sequence is present in the sample, the first and second probes will, under appropriate conditions, hybridize to adjacent regions on the target nucleic acid sequence (see, e.g., probes 2 and 3 hybridized to target nucleic acid sequence 1 in FIG. 2A). In FIG. 2A, the target nucleic acid sequence (1) is depicted as hybridized with a first probe (2), for illustration purposes shown here as comprising a 5′ primer-specific portion (25) and a target-specific portion (1 5a), and a second probe (3) comprising a 3′ primer-specific portion (35), a target-specific portion (15b) and a free 5′ phosphate group (“P”) for ligation.

In certain embodiments, the adjacently hybridized probes may, under appropriate conditions, be ligated together to form a ligation product (see, e.g., ligation product 6 in FIG. 2B). FIG. 2B depicts the ligation product (6), generated from the ligation of the first probe (2) and the second probe (3). The ligation product (6) is shown comprising the 5′ primer-specific portion (25) and the 3′ primer-specific portion (35). In certain embodiments, when the duplex comprising the target nucleic acid sequence (1) and the ligation product (6) is denatured, for example, by heating, the ligation product (6) is released.

In certain embodiments, one forms an amplification reaction composition comprising the ligation product 6, at least one primer set 7, a polymerase 8, and a double-stranded-dependent label (see, e.g., FIG. 2C). In certain embodiments, one carries out an amplification reaction with the amplification reaction composition and determines if the target nucleic acid is present in view of a determined Ct value. In certain embodiments, one carries out an amplification reaction with the amplification reaction composition and determines if there is a threshold difference in signal value during and/or after the amplification reaction to determine whether the target nucleic acid sequence is present.

In certain embodiments, if no target nucleic acid sequence had been present in the sample, no ligation product comprising the 5′ and 3′ primer-specfic portions would have been formed during the ligation reaction. Accordingly, there would not have been an appropriate Ct value and/or there would not have been a threshold difference in signal value, which would indicate the absence of target nucleic acid sequence in the sample. In certain embodiments, ligation products may form even if the appropriate target nucleic acid sequence is not in the sample, but such ligation occurs to a measurably lesser extent than when the appropriate target nucleic acid sequence is in the sample. In certain such embodiments, one can set an appropriate Ct value to differentiate between samples that include the appropriate target nucleic acid sequence and samples that do not include the appropriate target nucleic acid sequence. In certain such embodiments, one can set an appropriate threshold difference between detectable signal values to differentiate between samples that include the appropriate target nucleic acid sequence and samples that do not include the appropriate target nucleic acid sequence.

Certain embodiments may be substantially the same as those depicted in FIGS. 2A to 2C, except that two sets of ligation probes are used for detecting a given nucleic acid sequence. For example, in certain embodiments, the first set of ligation probes is the same as the set depicted in FIG. 2. In certain embodiments, the second set of ligation probes comprises a first probe that comprises a target-specific portion that hybridizes to the complement of the target nucleic acid sequence shown in FIG. 2. The first probe of the second set of ligation probes may have the same 5′ primer-specific portion as the first probe of the first set of ligation probes or may have a different 5′ primer-specific portion. In certain embodiments, the second set of ligation probes comprises a second probe that comprises a target-specific portion that hybridizes to the complement of the target nucleic acid sequence shown in FIG. 2. The second probe of the second set of ligation probes may have the same 3′ primer-specific portion as the second probe of the first set of ligation probes or may have a different 3′ primer-specific portion.

In certain embodiments, the initial target nucleic acid sequence is an RNA, and mRNA is used to generate a cDNA copy. In certain embodiments, the cDNA serves as a target nucleic acid sequence to which the first and second probes of the ligation probe set hybridize.

In certain embodiments, one may substantially remove unligated ligation probes prior to an amplification reaction. In certain embodiments, one may substantially remove unligated probes by using hybridization based pullout. In certain such embodiments, after the ligation reaction, one may expose the composition to a solid support that includes sequences complementary to at least a portion of at least one of the primer-specific portion, the target-specific portion, and another additional portion unique to the first probe of the ligation probe set. In certain embodiments, a substantial portion of any unligated second probes would not hybridize to the sequences of the solid support, and thus, would not be retained on the solid support.

In certain embodiments, one could then denature any ligation products and unligated first probes from the solid support. That denatured material could then be exposed to a second solid support that includes sequences complementary to at least a portion of at least one of the primer-specific portion, the target-specific portion, and another additional portion unique to the second probe of the ligation probe set. In certain embodiments, a substantial portion of any unligated first probes would not hybridize to the sequences of the solid support, and thus, would not be retained on the solid support. In certain embodiments, one could then denature the material from the second solid support and subject that material to an amplification reaction.

In this application, whenever one employs an amplification reaction to determine whether there is a threshold difference in signal value from a label, the amplification reaction is carried out in a manner that will result in such a threshold difference if the target sequence that is being sought is included in the sample. In this application, whenever one employs an amplification reaction to determine whether there is an appropriate time threshold value and/or an appropriate cycle threshold value signifying the presence of a target nucleic acid sequence, the amplification reaction is carried out in a manner that will result in such an appropriate time threshold value and/or an appropriate cycle threshold value if the target sequence that is being sought is included in the sample. The following nonlimiting exemplary embodiments illustrate this concept.

In certain embodiments, one employs a ligation probe set that comprises: a first probe that comprises a 5′ primer specific portion and a target-specific portion; and a second probe that comprises a target specific portion and a 3′ primer-specific portion. If the target nucleic acid is present in the sample, the first and second probes are ligated together to form a ligation product during a ligation reaction. The ligation product comprises the 5′ primer-specific portion, the two target-specific portions, and the 3′ primer-specific portion.

In certain embodiments, one forms an amplification reaction composition comprising the ligation product, a double-stranded-dependent label, and a set of appropriate primers for the 5′ and 3′ primer-specific portions. The double-stranded-dependent label has a first detectable signal value when it is not exposed to double-stranded nucleic acid sequences. In certain embodiments, PCR is used as the amplification reaction.

In certain embodiments, if unligated probes are not substantially removed from the amplification reaction composition prior to the first cycle of amplification, no threshold difference is detected during and/or after the first cycle. No threshold difference is detected in such embodiments, because, whether or not the sought ligation product is present. the first cycle of amplification will not result in sufficient detectable signal from the double-stranded-dependent label, since there will be insufficient double-stranded nucleic acid after just one cycle.

In certain embodiments, in one or more subsequent cycles, sufficient double-stranded nucleic acid will-be present that results in sufficient detectable signal. In certain such embodiments, a threshold difference in detectable signal value will result in such subsequent cycles of amplification when amplification products with both the 5′ primer-specific portion and the 3′ primer-specific portion increase exponentially when the ligation product is amplified. In such subsequent cycles, if no ligation product is present, such amplification products will only increase linearly from the presence of the unligated probes. Such linear amplification occurs, since, unlike the ligation product, the unligated probes do not comprise 5′ primer-specific portions.

In certain embodiments, a threshold difference in detectable signal value may result after one or more cycles of amplification if the system can detect a difference in signal based on the different lengths of the double-stranded nucleic acids. Specifically, in certain embodiments, the double-stranded-dependent label may result in a higher detectable signal value for longer length double-stranded nucleic acids than for shorter length double-stranded nucleic acids. For example, the double-stranded nucleic acid resulting from amplification of unligated primers are shorter than the double-stranded nucleic acid resulting from amplification of ligation products. In certain embodiments, a threshold difference in detectable signal value may result after one or more cycles of amplification in view of the different detectable signal values resulting from the different sizes of the double-stranded nucleic acids.

In certain embodiments, one may employ a positive control, which is a separate amplification reaction, that is known to contain the target nucleic acid sequence and which comprises the same probe set and primers as the sample being tested. In certain embodiments, one may employ a negative control, which is a separate amplification reaction, that is known not to contain the target nucleic acid sequence and which comprises the same probe set and primers as the sample being tested.

In certain embodiments, one may carry out the ligation reaction in a reaction volume that comprises all of the reagents for both the ligation and amplification reactions (“closed-tube” reactions). In certain such embodiments, one may then carry out the amplification reaction without removing ligation product from that reaction volume. Thus, in certain such embodiments, the reaction volume may comprise: the sample, a ligation probe set, a ligation agent, a polymerase, a double-stranded-dependent label, a primer set, and dNTPs.

In certain such embodiments, one may employ a ligation reagent that does not function at the higher temperatures employed in a subsequent amplification reaction. In certain embodiments, one may substantially destroy the ligation reagent activity after the ligation reaction by subjecting the reaction volume to a high temperature for a given period of time prior to the amplification reaction. For example, in certain embodiments, one may employ a high temperature for a short cycle period during a ligation reaction such that the ligation reagent activity is not substantially destroyed, and after the ligation reaction, hold the reaction volume at the high temperature for a longer period of time that destroys a substantial amount of the ligation reagent activity. In certain embodiments, destroying a substantial amount of ligation reagent activity means destroying at least 90% of the ligation reaction activity. In certain embodiments, at least 95% of the ligation reaction activity is destroyed. In certain embodiments, 100% of the ligation reaction activity is destroyed.

In certain embodiments, one may employ other methods of substantially destroying the ligation reagent activity prior to the subsequent amplification reaction. For example, one may employ an agent that inhibits the activity of a ligation reagent at a higher temperature that is used for an amplification reaction, but that does not inhibit the ligation reagent at a lower temperature that is used for the ligation reaction.

In certain embodiments in which one includes amplification reagents in the reaction volume during a ligation reaction, one may employ amplification primers that do not interfere with hybridization and ligation of ligation probes during the ligation reaction.

In certain embodiments in which one includes amplification reagents in the reaction volume during a ligation reaction, one may employ polymerase that is substantially inactive in the ligation conditions that are employed. In certain embodiments, substantially inactive means that at least 90% of the polymerase is inactive. In certain embodiments, at least 95% of the polymerase is inactive. In certain embodiments, 100% of the polymerase is inactive.

In certain such embodiments, the polymerase may be substantially inactive at the temperatures that are employed for the ligation reaction. For example, in certain embodiments, a polymerase may not be substantially active at a lower temperature that is employed for a ligation reaction and the ligation reagent is active at such lower temperatures. In certain embodiments, one may employ an agent that inhibits the activity of a polymerase at a lower temperature that is used for a ligation reaction, but that does not inhibit the polymerase at a higher temperature that is used in an amplification reaction. Exemplary agents that may be used in such embodiments to inhibit polymerases at a lower temperature include, but are not limited to, aptamers. See, e.g., Lin et al., J. Mol. Biol., 271:100-111 (1997).

In certain embodiments, one may employ a polymerase that is not substantially activated at the conditions employed for a ligation reaction, but is subsequently activated after the ligation reaction. For example, in certain such embodiments, one may employ a polymerase that is not substantially activated when held at a high temperature for a short period, but is activated if held at the high temperature for a longer period. Using such a polymerase according to certain embodiments, one may employ a high temperature for a short cycle period during a ligation reaction such that the polymerase is not substantially activated, and after the ligation reaction, hold the reaction volume at the high temperature for a longer period of time such that the polymerase is activated. An exemplary, but nonlimiting, example of such a polymerase is AmpliTaq Gold® (Applied Biosystems, Foster City, Calif.).

In certain embodiments in which one includes amplification reagents in the reaction volume during a ligation reaction, one may employ double-stranded-dependent labels that do not interfere with hybridization and ligation of ligation probes during the ligation reaction.

In certain embodiments, one may add some or all of the reagents for the amplification reaction directly to the ligation reaction volume after a ligation reaction (“open tube” reactions). In certain embodiments, one may add at least a portion of the ligation reaction volume after a ligation reaction to reagents for the amplification reaction.

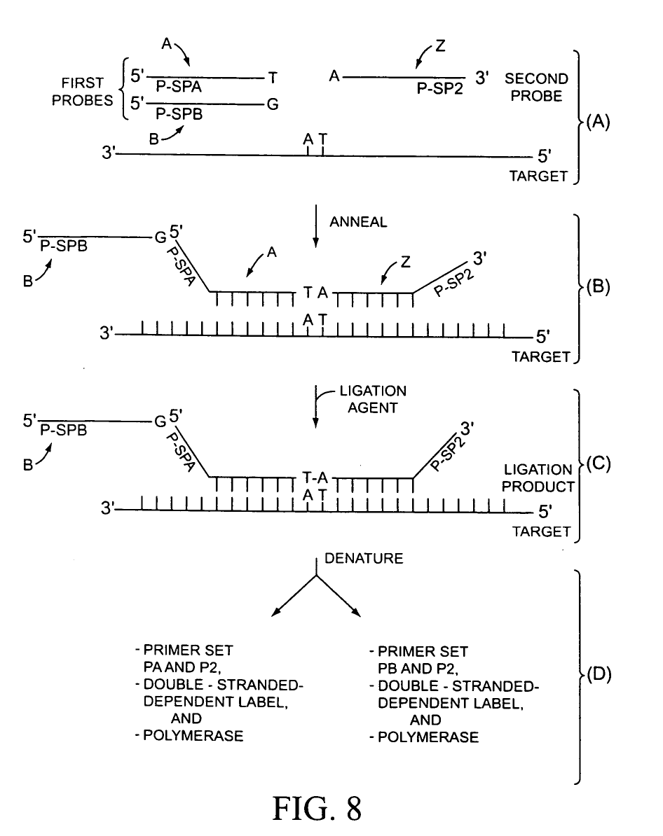

According to certain embodiments, the first and second probes in each ligation probe set are designed to be complementary to the sequences immediately flanking the pivotal nucleotide of the target sequence (see, e.g., probes A, B, and Z in FIG. 8(A)). In the embodiment shown in FIG. 8, two first probes A and B of a ligation probe set will comprise a different nucleotide at the pivotal complement and a different primer-specific portion (P-SPA and P-SPB, respectively) for each different nucleotide at the pivotal complement. One forms a ligation reaction composition comprising the probe set and the sample.

When the target sequence is present in the sample, the first and second probes will hybridize, under appropriate conditions, to adjacent regions on the target (see, e.g., FIG. 8(B)). When the pivotal complement is base-paired to the target, in the presence of an appropriate ligation agent, two adjacently hybridized probes may be ligated together to form a ligation product (see, e.g., FIG. 8(C)). In certain embodiments, if the pivotal complement of a first probe is not base-paired to the target, no ligation product comprising that mismatched probe will be formed (see, e.g., probe B in FIGS. 8(B) to 8(D).

In FIGS. 8(B) and 8(C), the first probe B is not hybridized to a target. In certain embodiments, the failure of a probe with a mismatched terminal pivotal complement to ligate to a second probe may arise from the failure of the probe with the mismatch to hybridize to the target under the conditions employed. In certain embodiments, the failure of a probe with a mismatched terminal pivotal complement to ligate to a second probe may arise when that probe with the mismatch is hybridized to the target, but the nucleotide at the pivotal complement is not base-paired to the target.

In certain embodiments, the reaction volume that is subjected to the ligation reaction forms a test composition. In certain embodiments, one then forms an amplification reaction composition comprising at least a portion of the test composition, a primer set comprising at least one primer comprising at least a portion of the sequence of one of the optional primer-specific portions P-SPA or P-SPB, a polymerase, and a double-stranded-dependent label (see, e.g., FIG. 8(D)).

In certain embodiments, in certain appropriate salts, buffers, and nucleotide triphosphates, the amplification reaction composition is subjected to an amplification reaction. In this example, no target nucleic acid sequence in the sample has a pivotal nucleotide (C) that is complementary to the nucleotide of the pivotal complement of probe B. Thus, in this example, no ligation product comprising both 5′ primer-specific portion P-SPB and the 3′ primer-specific portion P-SP2 is formed. Accordingly, in certain such embodiments, the amplification reaction comprising the primer set PB and P2 should result in a ΔCt that indicates that no target nucleic acid sequence is present. In certain embodiments, the amplification reaction comprising the primer set PB and P2 should result in no threshold difference in signal value, which indicates that no target nucleic acid sequence is present. In certain embodiments, ligation of probes with a pivotal complement that is not complementary to the pivotal nucleotide may occur, but such ligation occurs to a measurably lesser extent than ligation of probes with a pivotal complement that is complementary to the pivotal nucleotide. In certain such embodiments, one can set an appropriate ΔCt and/or an appropriate threshold difference between detectable signal values to differentiate between samples that include the appropriate target nucleic acid sequence and samples that do not include the appropriate target nucleic acid sequence.

In certain embodiments, to determine the presence or absence of he two optional target nucleic acid sequences, one can compare the Ct value of the amplification reaction employing the primer set PA and P2 to the Ct value of the amplification reaction employing the primer set PB and P2. For example, one may determine the ΔCt as follows:

ΔCt=Ct (amplification with primers PB and P2) minus Ct (amplification with primers PA and P2).

In certain embodiments, one can then set various ΔCt values to determine whether the sample is heterozygous or homozygous for one of the two alleles. For example, in certain embodiments, one may conclude that the sample: is homozygous for the pivotal nucleotide corresponding to probe A if the ΔCt is greater than or equal to 4.5; homozygous for the pivotal nucleotide corresponding to probe B if the ΔCt is less than or equal to −2; heterozygous if ΔCt is greater than or equal to −1 and less than or equal to 3.5; and make no call if ΔCt is greater than −2 and less than −1 or greater than 3.5 and less than 4.5. Also, in certain embodiments, one may conclude that there are no ligation products if the Ct of both amplification reactions is greater than the average Ct of a control (containing no DNA) minus two or more standard deviations. In various embodiments, one may set the ranges of ΔCt values at other levels as appropriate for determining the presence of absence of various alleles.

In certain embodiments, FIG. 8 can be modified to include an additional probe set for detecting the presence or absence of a nucleic acid sequence complementary to the target nucleic acid sequence sought to be detected in FIG. 8. Thus, the pivotal nucleotide of such a complementary target nucleic acid sequence in FIG. 8 will be either (T) or (G). Accordingly in certain embodiments, the first probes of the additional probe set comprise a target-specific portion complementary to a portion of the complementary target nucleic acid sequence and will have either (A) or (C) as the pivotal complement. For convenience in this example, the first probe with (A) as the pivotal complement is designated probe C, and the first probe with (C) as the pivotal complement is designated probe D. In certain embodiments, the first probes A and C may share the same primer-specific portion P-SPA, and the first probes B and D may share the same primer-specific portion P-SPB. In certain such embodiments, each of the two separate amplification reactions as shown in FIG. 8 would amplify the ligation products for one of the two different target nucleic sequences and its complement. In certain embodiments, each of the different probes A, B, C, and D may have different 5′ primer-specific portions, and four different amplification reactions with four different primer sets may be performed.

In certain embodiments, the methods of the invention comprise universal primers, universal primer sets, or both. In certain embodiments, one may use a single universal primer set for any number of amplification reactions for different target sequences.

The methods of the present invention according to certain embodiments may comprise universal primers or universal primer sets that decrease the number of different primers that are added to the reaction composition, reducing the cost and time required.

The skilled artisan will appreciate that in certain embodiments, including, but not limited to, detecting multiple alleles, the ligation reaction composition may comprise more than one first probe or more than one second probe for each potential allele in a multiallelic target locus.

In certain embodiments, one may employ the same two different primer-specific portions for the two different allelic options at more than one locus. In certain such embodiments, one may distinguish between the different loci by employing a different reaction composition for each locus.

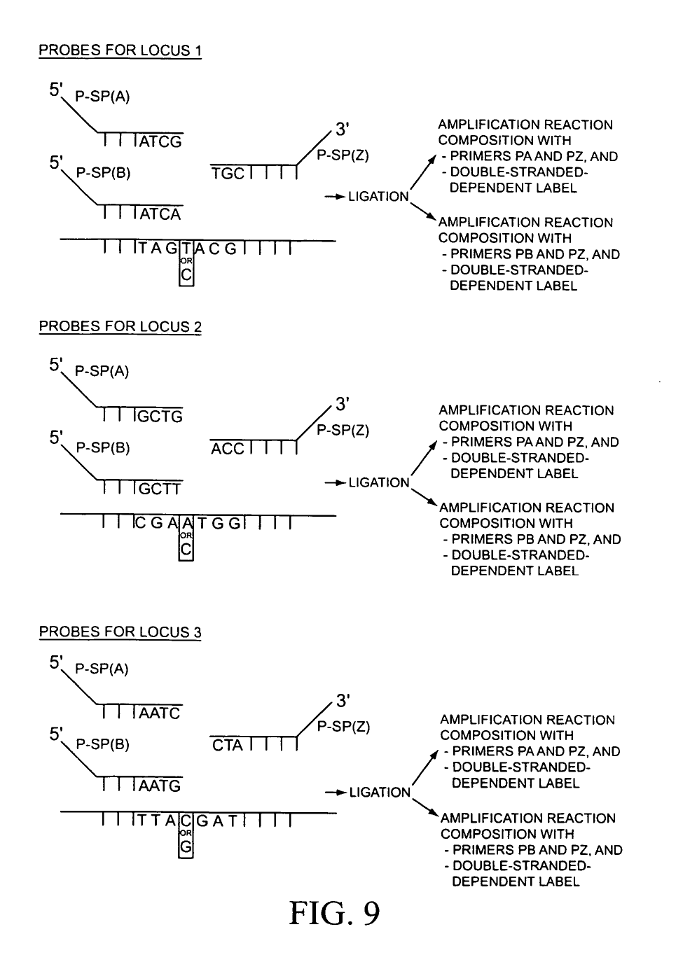

Thus, it one wants to determine a single nucleotide difference in the alleles at three different biallelic loci, in certain such embodiments, one may employ three different ligation reaction compositions that each has a different ligation probe set specific for the two options at each locus. FIG. 9 illustrates certain such embodiments in which one employs three different ligation reaction compositions for three biallelic loci. In FIG. 9, there is a different probe set for each of the three different loci. Each probe set comprises two first probes for the two different alleles at each locus. Each of the first probes of each probe set comprises a target-specific portion that is complementary to a portion of the given locus and includes a different nucleotide at the pivotal complement (A or G for the first locus; T or G for the second locus; G or C for the third locus), and a different 5′ primer-specific portion (P-SP(A) or P-SP(B)) corresponding to one of the two alleic nucleotide options for each locus. The same set of 5′ primer-specific portions (P-SP(A) or P-SP(B)) can be used on the two first probes of each of the three different probe sets. Each of the second probes of each probe set comprises the same 3′ primer-specific portion (P-SP(Z)) and a different target-specific portion for each different locus.

In certain embodiments shown in FIG. 9, after the separate ligation reactions for each of the three loci, one can perform six separate amplification reactions. In certain embodiments shown in FIG. 9, the material from each of the three separate ligation reactions is split into two separate amplification reactions; one with primer set (PA) and (PZ), and one with primer set (PB) and PZ). The amplification reactions each include a double-stranded-dependent label.

In certain such embodiments, one can determine the ΔCt value between the two separate amplification reactions for each locus to determine whether the sample is homozygous for one of the alleles or is heterozygous. In certain embodiments, one may determine whether there is a threshold difference in signal value for each of the six separate amplification reactions to determine for each locus whether the sample is homozygous for one of the alleles or is heterozygous.

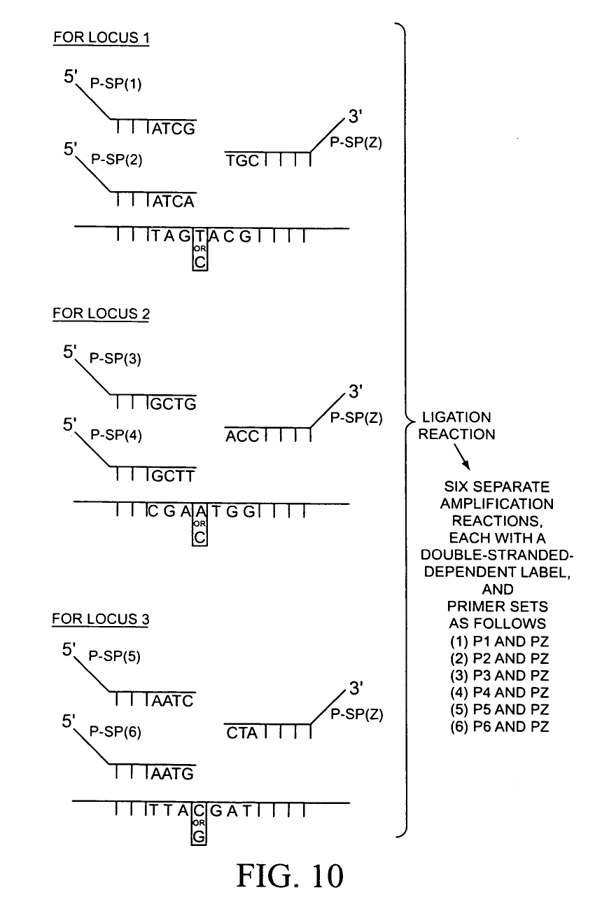

In certain embodiments, one may employ different probes with different primer-specific portions for each different allele at each locus. FIG. 10 illustrates certain such embodiments in which there are three biallelic loci. In FIG. 10, for each locus, one employs a ligation probe set comprising two first probes. In FIG. 10, there is a different probe set for each of the three different loci. Each probe set comprises two first probes for the two different alleles at each locus. Each of the first probes of each probe set comprises a target-specific portion that is complementary to a portion of the given locus and includes a different nucleotide at the pivotal complement (A or G for the first locus; T or G for the second locus; G or C for the third locus), and a different 5′ primer-specific portion (P-SP(1) and P-SP(2) for the first locus; P-SP(3) and P-SP(4) for the second locus; P-SP(5) and P-SP(6) for the third locus). Each of the second probes of each probe set comprises the same 3′ primer-specific portion (P-SP(Z)) and a different target-specific portion for each different locus.

In certain such embodiments, one can perform a ligation reaction with all of the probe sets for all of the loci. In certain embodiments shown in FIG. 10, after ligation, one can perform six separate amplification reactions, each with one of six different primer sets as follows: (1) primer set (P1) and (PZ); (2) primer set (P2) and (PZ); (3) primer set (P3) and (PZ); (4) primer set (P4) and (PZ); (5) primer set (P5) and (PZ); and (6) primer set (P6) and (PZ).

In certain such embodiments, one can determine the ΔCt value between the two separate amplification reactions for each locus to determine whether the sample is homozygous for one of the alleles or is heterozygous. In certain embodiments, one may determine whether there is a threshold difference in signal value for each of the six separate amplification reactions to determine for each locus whether the sample is homozygous for one of the alleles or is heterozygous.

The embodiment in FIG. 9 can be modified such that one performs six separate ligations reactions, one for each allele at each of the three loci. In certain such embodiments, each of the six separate ligation reactions has one of the six different first probes depicted in FIG. 9. In certain such embodiments, one may modify each of the six different first probes depicted in FIG. 9 by employing the same 5′ primer-specific portion on each of the six different probes, since each of those six different probes will be subjected to separate ligation reactions. In certain embodiments, each of the six separate ligation reactions includes the appropriate second probe for the particular locus.

In certain such embodiments employing six separate ligation reactions with different first probes, one may include in the composition prior to ligation, the appropriate primer set for the probe set, the double-stranded-dependent label, and other components for the subsequent amplification reaction. In certain embodiments employing six separate ligation reactions with different first probes, after the ligation reaction, one may add directly to the material subjected to ligation reaction the appropriate primer set for the probe set, the double-stranded-dependent label, and other components for the subsequent amplification reaction.

In certain embodiments, one may analyze many different target sequences employing specific different probe sets in separate reaction compositions. For example, one could employ a 96 well plate with 96 different ligation probe sets for 96 different target nucleic acid sequences. In certain embodiments, one may want to detect the presence or absence of (or to quantitate) a single target nucleic acid sequence with each of the 96 probe sets. In certain such embodiments, one may employ the same set of two primers and the same double-stranded-dependent label in each of the different 96 wells to obtain results for 96 different target sequences.

In certain embodiments, one may want to detect the presence or absence of (or to quantitate) two different alleles at 48 different loci with 96 different ligation probe sets. In certain embodiments, one employs two separate probe sets in two separate wells for each of the 48 different loci, and each probe set comprises a first probe and a second probe. In certain embodiments, each of the first probes of each of the two probe sets for each locus comprises a target-specific portion that is complementary to a portion of one of the 48 different loci and includes a different nucleotide at the pivotal complement. In certain embodiments, the second probes of the two probe sets for each locus are the same, and the second probes in probe sets for different loci are complementary to a portion of one of the 48 different loci. In certain embodiments, the two first probes of each of the 96 probe sets may further comprise the same primer-specific portion. In certain embodiments, each of the second probes of each of the 96 probe sets may further comprise another primer-specific portion.

In certain such embodiments, after ligation, one may perform 96 separate amplification reactions in the 96 different wells. In certain such embodiments, one may use in all of the 96 wells the same primer set and the same double-stranded-dependent label. One may detect which allele or alleles are present in each of 96 wells with appropriate ΔCt values and/or by detecting the presence or absence of an appropriate threshold difference in detectable signal values.

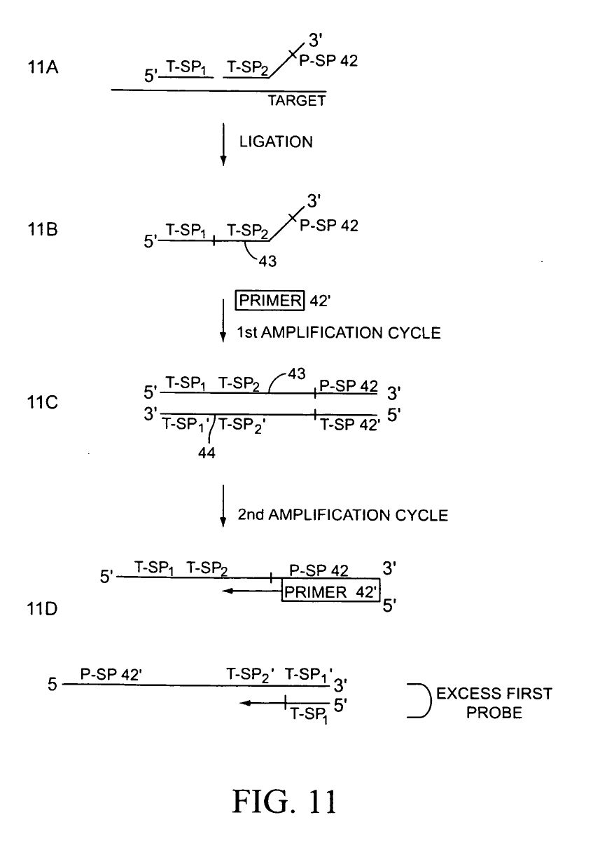

In certain embodiments, one may employ a ligation probe set that includes an excess of the first probe to serve as a primer in subsequent amplification reactions. FIG. 11 shows certain exemplary embodiments. In FIG. 11, the first probe comprises a target-specific portion T-SP1. The second probe comprises a 3′ primer-specific portion P-SP 42 and a target-specific portion T-SP2.

In such embodiments, after ligation (see FIGS. 11A and 11B), the primer set included in the amplification reaction composition may only comprise one primer 42′ that comprises a sequence that is complementary to the sequence of the 3′ primer-specific portion P-SP 42 of the second probe. After ligation, a cycle of amplification with that primer results in an amplification product that comprises a sequence complementary to the ligation product (see FIG. 11C).

In the second cycle of amplification, the primer P-SP 42′ again results in an amplification product that comprises a sequence complementary to the ligation product (see FIG. 11D). Moreover, excess first probe serves as a primer that interacts with the sequence that is complementary to the ligation product to form an amplification product that comprises the sequence of the ligation product (see FIG. 11D).

In certain embodiments, the first probe may contain additional nucleotides at the 5′ end that do not hybridize to the target nucleic acid sequence.

Certain embodiments that employ excess first probe as a primer for subsequent amplification reactions can be used in the various embodiments of ligation and amplification that are discussed throughout this application. Examples include, but are not limited to, the embodiments depicted in FIG. 7. According to certain such embodiments, one may modify the first probes Z that are shown in FIG. 7 by not including a primer-specific portion P-SP1. In a subsequent amplification reaction, one may employ excess first probes to serve as primers rather than employing primers that correspond to a P-SP1 sequence on the first probe shown in FIG. 7.

The skilled artisan will understand that, in various embodiments, ligation probes can be designed with a pivotal complement at any location in either the first probe or the second probe. Additionally, in certain embodiments, ligation probes may comprise multiple pivotal complements.

In certain embodiments that employ ligation probe sets that comprise multiple first probes for a given locus that comprise target-specific portions with different pivotal complements, the target-specific portions of each of the different first probes for a given locus may have the same sequence except for a different nucleotide at the pivotal complement. In certain embodiments, the target-specific portions of each of the first probes for a given locus may have a different nucleotide at the pivotal complement and may have different length sequences 5′ to the pivotal complement. In certain such embodiments, such target-specific portion sequences 5′ to the pivotal complement may all be complementary to a portion of the same locus nucleic acid sequence adjacent to the pivotal nucleotide, but may have different lengths. For example, in such embodiments in which there are two different first probes, the target-specific portion sequences 5′ to the pivotal complement may be the same except one of them may have one or more additional nucleotides at the 5′ end of the target-specific portion.

In certain embodiments that employ ligation probe sets that comprise multiple second probes for a given locus that comprise target-specific portions with different pivotal complements, the target-specific portions of each of the different second probes for a given locus may have the same sequence except for a different nucleotide at the pivotal complement. In certain embodiments, the target-specific portions of each of the second probes for a given locus may have a different nucleotide at the pivotal complement and may have different length sequences 3′ to the pivotal complement. In certain such embodiments, such target-specific portion sequences 3′ to the pivotal complement may all be complementary to a portion of the same locus nucleic acid sequence adjacent to the pivotal nucleotide, but may have different lengths. For example, in such embodiments in which there are two different second probes, the target-specific portion sequences 3′ to the pivotal complement may be the same except one of them may have one or more additional nucleotides at the 3′ end of the target-specific portion.

In certain embodiments, one may add additional nucleotides to the end of a target specific portion of a ligation probe to affect its melting temperature. For example, in certain embodiments, the different nucleotide at the pivotal nucleotide of two first probes of a ligation probe set may result in different melting temperatures for such probes if they have the same length target-specific portion. In certain such embodiments. one may minimize such melting temperature differences by adding one or more additional nucleotides to the end of target-specific portion opposite the end that aligns with an adjacent ligation probe of a probe set.

In certain embodiments, one may employ probes that include one or more spacer nucleotides between a primer-specific portion and a target-specific portion. In certain embodiments, such a spacer nucleotide may be included to affect the melting temperature of a ligation probe. For example, in certain embodiments, one or more nucleotides of a primer-specific portion may be complementary to the target nucleic acid sequence in the region adjacent to the sequence that hybridizes to the target-specific portion of a ligation probe. For example, the end of a target-specific portion (TSP) adjacent to a primer-specific portion (PSP), and the end of the primer-specific portion adjacent to the target-specific portion may hybridize to a target nucleic acid as follows:

PSP/TSP (hybridizing portions shown
with double underlining)

.....ACG/ATC.....(ligation probe)
.....TGC/TAG.....(target nucleic acid)

In certain such embodiments, the hybridization of the one or more nucleotides of the primer-specific portion to the target influences the melting temperature of the probe.

In certain such embodiments, one may introduce one or more spacer nucleotides between the primer-specific portion and the target-specific portion of the probe such that the spacer nucleotide(s) and the primer-specific portion will not hybridize to the target nucleic acid. In the specific example above, for example, one may introduce a spacer “C” between the target-specific portion and the primer-specific portion as follows:

PSP/ /TSP (hybridizing portions shown
with double underlining)

...ACG/C/ATC.....(ligation probe)
.... TGC/TAG.....(target nucleic acid)

In certain embodiments, one or more spacer nucleotides may be included between different portions of a ligation probe. For example, in certain embodiments, one or more spacer nucleotides may be included between a primer-specific portion and a target-specific portion.

In certain embodiments, one or more ligation probes may include an addressable portion or an addressable support-specific portion as discussed, e.g., in U.S. Pat. No. 6,027,889, PCT Published Patent Application No. WO 01/92579, and U.S. patent application Ser. Nos. 09/584,905; 10/011,993; and 60/412,225.

In certain embodiments, the target-specific portions of two ligation probes that are intended to hybridize to the same portion of a target nucleic acid sequence may include different nucleotides as long as such differences do not prevent appropriate ligation. For example, in certain embodiments, as long as appropriate ligation is not prevented, two probes that comprise target-specific portions that are designed to hybridize to an identical portion of a target, but have different pivotal complements A and C at their 3′ ends, may include variation within the target-specific portion as follows (see lower case nucleotide):

5′ CATGCcAATGACGGA-3′
5′ CATGCgAATGACGGC-3′

In certain embodiments, the number of ligation probes used to detect any number of target sequences, is the product of the number of targets to be detected times the number of alleles to be detected per target plus one (i.e., (number of target sequences x [number of alleles +1]). Thus, to detect 3 biallelic sequences, for example, nine probes are used (3×[2+1]). In certain embodiments, to detect 4 triallelic sequences, 16 probes are used (4×[3+1]), and so forth.

The significance of the decrease in the number of primers and labels in certain embodiments, and therefore the cost and number of manipulations, becomes readily apparent when performing genetic screening of an individual for a large number of multiallelic loci or of many individuals. In certain embodiments, to amplify the ligation product of a target sequence, two primers are used. One primer is complementary to the sequence of the 3′ primer-specific portion of the ligation products, and one primer comprises the sequence of the 5′ primer-specific portion. Using certain conventional methods, one employs three different primers for each different ligation product. Thus, to amplify the ligation products for three biallelic loci potentially present in an individual using certain conventional methodology, one would use 9 (3n, where n=3) primers.

In contrast, certain embodiments of the present invention can effectively reduce this number to as few as one amplification primer. According to certain embodiments of the present invention, as few as two “universal” primers, can be used to amplify one or more ligation or amplification products, since the probes may be designed to share primer-specific portions. A sample containing 100 possible bialielic loci would require 200 primers in certain conventional detection methods, yet only one universal primer can be used in certain embodiments of the present invention.

Also, in certain embodiments, one may prescreen a sample for the presence or absence of certain sequences. For example, in certain embodiments, one may employ different ligation probes sets to detect nucleotides at different loci. If the appropriate Ct value is not attained and/or if no threshold difference in detectable signal value is detected, one concludes that the sample is negative for all of the sequences in question. If the appropriate Ct value is attained and/or if there is a threshold difference in detectable signal value during or after an amplification reaction, one concludes that at least one of the sequences in question is present. In certain such embodiments, one could further screen the sample to determine which specific sequence(s) are present.

E. Certain Exemplary Applications

According to certain embodiments, the present invention may be used to detect the presence or absence of (or to quantitate) splice variants in a target nucleic acid sequence. For example, genes, the DNA that encodes for a protein or proteins, may contain a series of coding regions, referred to as exons, interspersed by non-coding regions referred to as introns. In a splicing process, introns are removed and exons are juxtaposed so that the final RNA molecule, typically a messenger RNA (mRNA), comprises a continuous coding sequence. While some genes encode a single protein or polypeptide, other genes can code for a multitude of proteins or polypeptides due to alternate splicing.

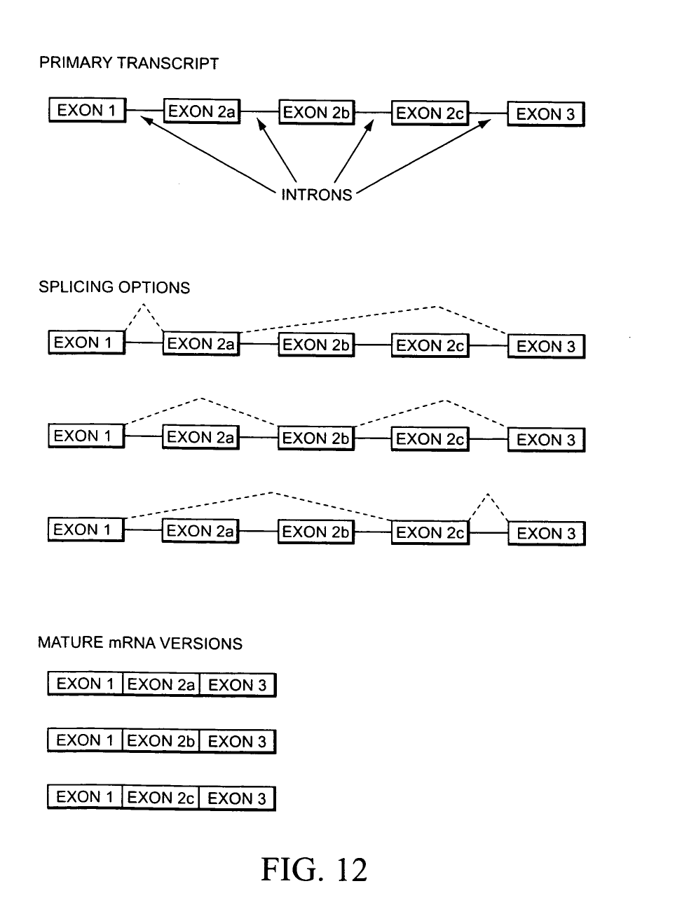

For example, a gene may comprise five exons each separated from the other exons by at least one intron, see FIG. 12. The hypothetical gene that encodes the primary transcript, shown at the top of FIG. 12, codes for three different proteins, each encoded by one of the three mature mRNAs, shown at the bottom of FIG. 12. Due to alternate splicing, exon 1 may be juxtaposed with (a) exon 2a-exon 3, (b) exon 2b-exon 3, or (c) exon 2c-exon 3, the three splicing options depicted in FIG. 12, which result in the three different versions of mature mRNA.

The rat muscle protein, troponin T is but one example of alternate splicing. The gene encoding troponin T comprises five exons (W, X, α, β, and Z), each encoding a domain of the final protein. The five exons are separated by introns. Two different proteins, an α-form and a β-form are produced by alternate splicing of the troponin T gene. The α-form is translated from an mRNA that contains exons W, X, α, and Z. The β-form is translated from an mRNA that contains exons W, X, β, and Z.

Certain exemplary embodiments involving splice variants follow. In this application, the use of the terms “first exon” and “second exon” are not limited to the actual first exon and the actual second exon of a given nucleic acid sequence, unless such terms are explicitly used in that manner. Rather, those terms are used to differentiate between any adjoining exons. Thus, one may want to distinguish between two different splice variants of Sequence A, one of which comprises Exons 2 and 3 of Sequence A and one of which comprises Exons 2 and 5 of Sequence A. In the embodiments discussed herein, Exon 2 of Sequence A would be the “first exon” and Exons 3 and 5 of Sequence A would be two “second exons.”

In certain embodiments, a method is provided for detecting the presence or absence of (or quantitating) at least one splice variant of at least one given nucleic acid sequence in a sample, wherein the at least one splice variant comprises a sequence that corresponds to a juncture between a first exon and one of a plurality of second exons. In certain embodiments, the method comprises forming a ligation reaction composition comprising the sample and a ligation probe set for each given nucleic acid sequence. In certain embodiments, the ligation probe set for each given nucleic acid sequence comprises: (1) a first probe that comprises (a) a target-specific portion that is complementary to a portion of the given nucleic acid sequence that corresponds to a portion of the first exon and (b) a 5′ primer-specific portion, and (2) at least one a second probe that comprises: (a) a splice-specific portion that is complementary to a portion of the given nucleic acid sequence that corresponds to a portion of one of the plurality of second exons; (b) a 3′ primer-specific portion, wherein the 3′ primer-specific portion is specific for the one of the plurality of second exons.

If the sample comprises a sequence corresponding to the juncture of the first exon and the one of the plurality of second exons, the first probe and the second probe, which comprises the splice-specific portion that is complementary to the portion of the given nucleic acid sequence that corresponds to the portion of the one of the plurality of second exons, hybridize to the given nucleic acid sequence adjacent to one another so that they are suitable for ligation together.

In certain embodiments, one forms a test composition by subjecting the ligation reaction composition to at least one cycle of ligation, wherein adjacently hybridized probes are ligated together to form a ligation product comprising the 5′ primer-specific portion, the target-specific portion, the splice-specific portion, and the 3′ primer-specific portion.

In certain embodiments, one forms an amplification reaction composition comprising: (1) the test composition; (2) a polymerase; (3) at least one double-stranded-dependent label, wherein the at least one double-stranded-dependent label has a first detectable signal value when it is not exposed to double-stranded nucleic acid sequence; and (4) a primer set comprising at least one first primer comprising the sequence of the 5′ primer-specific portion of the ligation product and at least one second primer comprising a sequence complementary to the sequence of the 3′ primer-specific portion of the ligation product.

In certain embodiments, one subjects the amplification reaction composition to an amplification reaction. In certain embodiments, one detects a second detectable signal value from the at least one double-stranded-dependent label at least one of during and after the amplification reaction. In certain embodiments, a threshold difference between the first detectable signal value from the at least one double-stranded-dependent label and the second detectable signal value from the at least one double-stranded-dependent label indicates the presence of the at least one splice variant of the at least one given target nucleic acid sequence. In such embodiments, no threshold difference between the first detectable signal value from the at least one double-stranded-dependent label and the second detectable signal value from the at least one double-stranded-dependent label indicates the absence of the at least one splice variant of the at least one given target nucleic acid sequence. In certain embodiments, one may employ Ct values to determine the presence or absence of the at least one splice variant of the at least one given target nucleic acid sequence.

In certain embodiments, one may desire to detect the presence or absence of (or to quantitate) more than one splice variant of a given nucleic acid sequence. In certain such embodiments, one may employ multiple second probes each comprising a different splice-specific sequence and a different primer-specific portion for each different second exon sought to be detected or quantitated. In certain such embodiments, one may employ separate amplification reactions with different appropriate primer sets for the different second probes.

In certain embodiments, the quantity of the at least one splice variant in the at least one target nucleic acid sequence is determined.

In certain embodiments, a method is provided for detecting the presence or absence of (or quantitating) at least one splice variant of at least one given nucleic acid sequence in a sample comprising forming a ligation reaction composition comprising the sample and a ligation probe set for each given nucleic acid sequence. In certain embodiments, the ligation probe set for each given nucleic acid sequence comprises: (1) at least one first probe that comprises: (a) a 5′ primer-specific portion, and (b) a splice-specific portion that is complementary to a portion of the given nucleic acid sequence that corresponds to a portion of one of the plurality of second exons, wherein the 5′ primer-specific portion is specific for the one of the plurality of second exons; and (2) a second probe that comprises: (a) a target-specific portion that is complementary to a portion of the given nucleic acid sequence that corresponds to the first exon and (b) a 3′ primer-specific portion.

If the target nucleic acid comprises a sequence corresponding to the juncture of the first and second exon, the first and second probe of the probe set hybridize to the given nucleic acid sequence adjacent to one another so that they are suitable for ligation together.

In certain embodiments, one forms a test composition by subjecting the ligation reaction composition to at least one cycle of ligation, wherein adjacently hybridized probes are ligated together to form a ligation product comprising the 5′ primer-specific portion, the splice-specific portion, the target-specific portion, and the 3′ primer-specific portion.

In certain embodiments, one forms an amplification reaction composition comprising: (1) the test composition; (2) a polymerase; (3) at least one double-stranded-dependent label, wherein the double-stranded-dependent label has a first detectable signal value when it is not exposed to double-tranded nucleic acid sequence; and (4) a primer set comprising at least one first primer comprising the sequence of the 5′ primer-specific portion of the ligation product and at least one second primer comprising a sequence complementary to the sequence of the 3′ primer-specific portion of the ligation product.

In certain embodiments, one subjects the amplification reaction composition to an amplification reaction. In certain embodiments, one detects a second detectable signal value from the at least one double-stranded-dependent label at least one of during and after the amplification reaction. In certain embodiments, a threshold difference between the first detectable signal value from the at least one double-stranded-dependent label and the second detectable signal value from the at least one double-stranded-dependent label indicates the presence of the at least one splice variant of the at least one given target nucleic acid sequence. In such embodiments, no threshold difference between the first detectable signal value from the at least one double-stranded-dependent label and the second detectable signal value from the at least one double-stranded-dependent label indicates the absence of the at least one splice variant of the at least one given target nucleic acid sequence. In certain embodiments, one may employ Ct values to determine the presence or absence of the at least one splice variant of the at least one given target nucleic acid sequence.

In certain embodiments, one may desire to detect the presence or absence of (or to quantitate) more than one splice variant of a given nucleic acid sequence. In certain such embodiments, one may employ multiple first probes each comprising a different splice-specific sequence and a different primer-specific portion for each different second exon sought to be detected or quantitated. In certain such embodiments, one may employ separate amplification reactions with different appropriate primer sets for the different first probes.

In certain embodiments, the quantity of the at least one splice variant in the at least one target nucleic acid sequence is determined.

In certain embodiments, the at least one target nucleic acid sequence comprises at least one complementary DNA (cDNA) generated from an RNA. In certain embodiments, the at least one cDNA is generated from at least one messenger RNA (mRNA). In certain embodiments, the at least one target nucleic acid sequence comprises at least one RNA target sequence present in the sample.

In various embodiments for detecting the presence or absence of (or quantitating) splice variants, one can use any of the various embodiments disclosed in this application. In various embodiments, either the first probe or the second probe or both may comprise splice specific portions for detecting the presence or absence of (or to quantitate) different splice variants. Also, in certain embodiments, if one desires to identify and quantify but one splice variant, they can use only one probe that comprises a splice-specific portion (specific to that one splice variant).

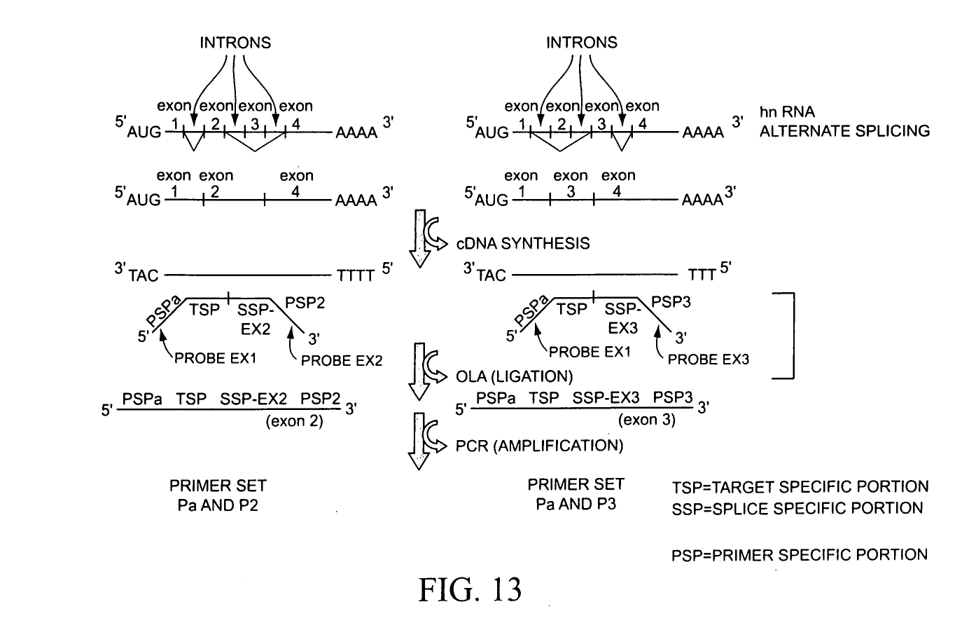

Certain nonlimiting embodiments for identifying splice variants are illustrated by FIG. 13. With such embodiments, one detects the presence or absence of (or quantitates) two different splice variants. One splice variant includes exon 1, exon 2, and exon 4. The other splice variant includes exon 1, exon 3, and exon 4.

In the depicted embodiments, one employs a ligation probe set that comprises a first probe (Probe EX1) that comprises a 5′ primer-specific portion (PSPa) and a target-specific portion that corresponds to at least a portion of exon 1 (TSP). The probe set further comprises two different second probes (Probe EX2 and Probe EX3). Probe EX2 comprises a 3′ primer-specific portion PSP2, and a splice-specific portion (SSP-EX2) that corresponds to at least a portion of exon 2. Probe EX3 comprises a 3′ primer-specific portion PSP3, and a splice-specific portion (SSP-EX3) that corresponds to at least a portion of exon 3.

In the embodiments depicted in FIG. 13, if a splice variant is present, the first and second probes corresponding to that splice variant hybridize adjacent to one another and are ligated together to form a ligation product. In the embodiments depicted in FIG. 13, two separate amplification reactions using a double-stranded-dependent label are performed; one with the primer set Pa and P2; and one with the primer set Pa and P3.

Thus, in FIG. 13, one concludes from the amplification reactions that ligation products corresponding to both exon 2 and exon 3 are present. With such results, one concludes that the sample comprises both splice variants.

In certain embodiments, when the gene expression levels for several target nucleic acid sequences for a sample are known, a gene expression profile for that sample can be compiled and compared with other samples. For example, but without limitation, samples may be obtained from two aliquots of cells from the same cell population, wherein one aliquot was grown in the presence of a chemical compound or drug and the other aliquot was not. By comparing the gene expression profiles for cells grown in the presence of drug with those grown in the absence of drug, one may be able to determine the drug effect on the expression of particular target genes.

In certain embodiments, one may quantitate the amount of mRNA encoding a particular protein within a cell to determine a particular condition of an individual. For example, the protein insulin, among other things, regulates the level of blood glucose. The amount of insulin that is produced in an individual can determine whether that individual is healthy or not. Insulin deficiency results in diabetes, a potentially fatal disease. Diabetic individuals typically have low levels of insulin mRNA and thus will produce low levels of insulin, while healthy individuals typically have higher levels of insulin mRNA and produce normal levels of insulin.

Another human disease typically due to abnormally low gene expression is Tay-Sachs disease. Children with Tay-Sachs disease lack, or are deficient in, a protein(s) required for sphingolipid breakdown. These children, therefore, have abnormally high levels of sphingolipids causing nervous system disorders that may result in death.

In certain embodiments, it is useful to identity and detect additional genetic-based diseases/disorders that are caused by gene over- or under-expression. Additionally, cancer and certain other known diseases or disorders may be detected by, or are related to, the over- or under-expression of certain genes. For example, men with prostate cancer typically produce abnormally high levels of prostate specific antigen (PSA); and proteins from tumor suppressor genes are believed to play critical roles in the development of many types of cancer.

Using nucleic acid technology, in certain embodiments, minute amounts of a biological sample can typically provide sufficient material to simultaneously test for many different diseases, disorders, and predispositions. Additionally, there are numerous other situations where it would be desirable to quantify the amount of specific target nucleic acids, in certain instances mRNA, in a cell or organism, a process sometimes referred to as “gene expression profiling.” When the quantity of a particular target nucleic acid within, for example, a specific cell-type or tissue, or an individual is known, in certain cases one may start to compile a gene expression profile for that cell-type, tissue, or individual. Comparing an individual's gene expression profile with known expression profiles may allow the diagnosis of certain diseases or disorders in certain cases. Predispositions or the susceptibility to developing certain diseases or disorders in the future may also be identified by evaluating gene expression profiles in certain cases. Gene expression profile analysis may also be useful for, among other things, genetic counseling and forensic testing in certain cases.

F. Certain Exemplary Kits

In certain embodiments, the invention also provides kits designed to expedite performing certain methods. In certain embodiments, kits serve to expedite the performance of the methods of interest by assembling two or more components used in carrying out the methods. In certain embodiments, kits may contain components in pre-measured unit amounts to minimize the need for measurements by end-users. In certain embodiments, kits may include instructions for performing one or more methods of the invention. In certain embodiments, the kit components are optimized to operate in conjunction with one another.

In certain embodiments, kits for detecting at least one target nucleic acid sequence in a sample are provided. In certain embodiments, the kits comprise:

(a) a ligation probe set for each target nucleic acid sequence, the probe set comprising

(i) at least one first probe, comprising a target-specific portion, a 5′ primer-specific portion, wherein the 5′ primer-specific portion comprises a sequence, and

(ii) at least one second probe, comprising a target-specific portion, a 3′ primer-specific portion, wherein the 3′ primer-specific portion comprises a sequence,

wherein the probes in each set are suitable for ligation together when hybridized adjacent to one another on a complementary target nucleic acid sequence; and

(b) a double-stranded-dependent label.

In certain embodiments, kits for detecting at least one target nucleic acid sequence in a sample are provided. In certain embodiments, the kits comprise:

(a) a ligation probe set for each target nucleic acid sequence, the probe set comprising

(i) at least one first probe, comprising a target-specific portion, a 5′ primer-specific portion, wherein the 5′ primer-specific portion comprises a sequence, and

(ii) at least one second probe, comprising a target-specific portion, a 3′ primer-specific portion, wherein the 3′ primer-specific portion comprises a sequence,

wherein the probes in each set are suitable for ligation together when hybridized adjacent to one another on a complementary target nucleic acid sequence; and

(b) a buffer comprising poly-deoxy-inosinic-deoxy-cytidylic acid.

In certain embodiments, compositions for a ligation reaction comprising a ligase and poly-deoxy-inosinic-deoxy-cytidylic acid are provided.

In certain embodiments, kits further comprise primers. In certain embodiments, kits further comprise at least one primer set comprising (i) at least one first primer comprising the sequence of the 5′ primer-specific portion of the at least one first probe, and (ii) at least one second primer comprising a sequence complementary to the sequence of the 3′ primer-specific portion of the at least one second probe.

In certain embodiments, kits comprise one or more additional components, including, without limitation, at least one of: at least one polymerase, at least one transcriptase, at least one ligation agent, oligonucleotide triphosphates, nucleotide analogs, reaction buffers, salts, ions, and stabilizers. In certain embodiments, kits comprise one or more reagents for purifying the ligation products, including, without limitation, at least one of dialysis membranes, chromatographic compounds, supports, and oligonucleotides.

The following examples are intended for illustration purposes only, and should not be construed as limiting the scope of the invention in any way.

### EXAMPLE 1

The following Table 1 is referred to throughout the following Example 1:

TABLE 1

Probe Set For Assay 1

| | |
|---|---|
| ASO1: | 5′ TGATGCTACTGGATCGCTGAAAGCACATTCCTCG3′ |
| ASO2: | 5′ TTGCCTGCTCGACTTAGAAAGCACATTCCTCA3′ |
| LSO: | 5′ Phosphate-GTCTTTGTTAAGTGCAGGAGCGCAAATCCGTATAGCCAAAGTGGTATCACTGGATAGCGACGT3′ |

Probe Set For Assay 2

| | |
|---|---|
| ASO1: | 5′ TGATGCTACTGGATCGCTGCCCATACACTGAGAC3′ |
| ASO2: | 5′ TTGCCTGCTCGACTTAGAGCCCATACACTGAGAT3′ |
| LSO: | 5′ Phosphate-GCTCCATATTGATTTATTTCCGAGTCGGACAATCCTGCGTTACATCACTGGATAGCGACGT3′ |

Probe Set For Assay 3

| | |
|---|---|
| ASO1: | 5′ TGATGCTACTGGATCGCTAGCTTTAAAACATTTTGTTGTATA3′ |
| ASO2: | 5′ TTGCCTGCTCGACTTAGACTTTAAAACATTTTGTTGTATG3′ |
| LSO: | 5′ Phosphate-TAGTTCAGATCTTGTAATAGATTGCCACCTTGGAACTGCGATCACTGGATAGCGACGT3′ |

DNAs
Three different genomic DNAs were purchased from Coriell Cell
Repositories (Coriell Institute for Medical Research, 403 Haddon
Avenue,Camden, NJ 08103):
NA17140
NA17155
NA17202
Universal PCR primer sequences

| | |
|---|---|
| UA1: | 5′TGATGCTACTGGATCGCT3′ |
| UA2: | 5′TTGCCTGCTCGACTTAGA3′ |
| UL: | 5′ACGTCGCTATCCAGTGAT3′ |

A. Ligation Probes

In these examples, a ligation probe set for each target nucleic acid sequence comprised first and second ligation probes designed to adjacently hybridize to the appropriate target nucleic acid sequence. These adjacently hybridized probes were, under appropriate conditions, ligated to form a ligation product.

This illustrative embodiment used three different ligation probe sets for detecting three biallelic loci. Three different samples of genomic DNA were tested. Table 1 shows the three probe sets that were used. The ligation probes included a target-specific portion, shown with underlined letters in Table 1. As shown by bold letters in Table 1, the ligation probes also included primer-specific portion sequences. Each probe set included two ASO (allele-specific oligo) probes, ASO1 and ASO2, which included a different nucleotide at the 3′ end to differentiate between the two different alleles at the given locus. Each probe set also included an LSO (locus-specific oligo) probe for the given locus.

The ligation probes were synthesized using conventional automated DNA synthesis chemistry.

B. Exemplary Ligation Reactions (Oligonucleotide Ligation Assay “OLA”)

Ligation reactions were performed in separate reaction volumes with each of the three different ligation probe sets shown in Table 1. The ligation reactions were performed in 96-well microtiter plates in 10 μL volumes with 2 nM (20 fmol) of each ASO probe (ASO1 and ASO2), 4 nM (40 fmol) of LSO probe, 0.12 units/μL (1.2 units) Taq Ligase (New England Biolabs, Inc., Beverly, Mass.), 10 ng/μL genomic DNA (100 ng/reaction) (partially fragmented by boiling for 15 minutes at 99° C. to an average size of 2 kb), and 1×ligation buffer (10×OLA Buffer Mixture: 200 mM Sodium (3-[N-Morpholino]propanesulfonate) (MOPS), pH 7.5 at 50° C., 1% (w/v) Triton X-100, 10 mM Dithiothreitol (DTT), 70 mM Magnesium Chloride, 2.5 mM Nicotinamide Adenine Dinucleotide (NAD), 300 ng/μL Poly [d(I-C)]).

Eight ligation control (LC) reactions that contain no genomic DNA were included for each 96-well microtiter plate.

For these examples, each of the three different probe sets in Table 1 were included in different reactions for three different genomic DNA samples. Thus, there were nine different ligation reaction volumes (not including the LC reactions), each with a different combination of probe set and genomic DNA sample. The three genomic DNA samples were obtained from Coriell Cell Repositories (Camden, N.J.) and were designated as follows: NA17140, NA17155, and NA17202.

The ligation reaction volumes were subjected to the reaction conditions shown in Table 2 below using an ABI GeneAmp® PCR System 9700 Thermal Cycler (Applied Biosystems, Foster City, Calif.). The ligation reaction volumes were chilled until they were transferred for the amplification reaction. The ligation reaction tubes were transferred to an ABI PRISM® 7900HT Sequence Detection System (Applied Biosystems, Foster City, Calif.) for amplification when the system reached the first hold temperature of 90° C.

TABLE 2

| Step | Step Type | Temperature (° C.) | Time |
|---|---|---|---|
| 1 | Hold | 90 | 3 minutes |
| 2 | 14 cycles | 90 | 5 seconds |
| | | 50 | 4 minutes |
| 3 | Hold | 99 | 10 minutes |
| 4 | HoId | 4 | ∞ |

C. Exemplary Amplification Reactions

One μL aliquots of each ligation reaction volume were amplified in two separate 15 μL PCR reactions with 7.5 μL SYBR® Green Master Mix (P/N 4309155, Applied Biosystems, Foster City, Calif.). One of the two separate PCR reactions included 500 nM (1.5 μmol total amount) of the universal primer UA1 and 500 nM (1.5 μmol total amount) of the universal primer UL that amplifies ligation products for allele 1; and the other of the two separate PCR reactions included 500 nM (1.5 μmol total amount) of the universal primer UA2 and 500 nM (1.5 μmol total amount) of the universal primer UL that amplifies ligation products for allele 2. SYBR® Green Master Mix includes SYBR® Green, PCR buffer, dNTPs, MgCl₂, and TaqGold® polymerase. SYBR® Green Master Mix contains dUTP instead of dTTP to allow AmpErase® Uracil N-glycosylase (UNG) digestion prior to each new PCR reaction to reduce carryover contamination. UNG (P/N N8080096 Applied Biosystems, Foster City, Calif.) was added to the reaction mixture at 0.1 unit/μL.

Each PCR reaction volume was subjected to reaction conditions shown in Table 3 below using an ABI PRISM® 7900HT Sequence Detection System (Applied Biosystems, Foster City, Calif.).

TABLE 3

| Step | Step Type | Temperature (° C.) | Time |
|---|---|---|---|
| 1 | Hold | 50 | 5 minutes |
| 2 | Hold | 95 | 12 minutes |
| 3 | 40 cycles | 95 | 5 seconds |
| | | 60 | 30 seconds |
| | | 72 | 30 seconds |

Product amplification was monitored in real-time through SYBR® Green I dye fluorescence utilizing the ABI PRISM® 7900HT Sequence Detection System (Applied Biosystems, Foster City, Calif.).

D. Exemplary Data Analysis

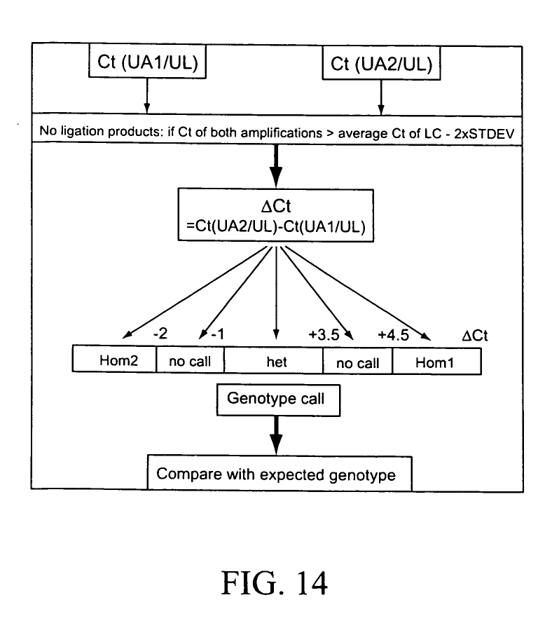

Genotype calls were made based on the allele-specific amplification rates monitored real-time by SYBR® Green I fluorescence (See FIG. 14). Threshold cycle (Ct) values were used as a measure for the input amount of allele 1 or allele 2 specific ligation product. The Ct value was the minimum number of cycles that resulted an intensity measurement of 1.

The reactions were tested for background ligation by comparing the Ct values of the reactions including genomic DNA to the Ct values of the ligation control reactions containing no gDNA (LC). Sufficient specific ligation product for genotype determination was determined to have been formed if for at least one PCR reaction of an amplification reaction pair (one SNP, one genomic DNA, two separate primer combinations) the Ct value is lower than the average Ct values of ligation control (LC) reactions minus 2 standard deviations.

Delta Ct values (αCt) were determined as follows:

ΔCt=Ct (amplification with UA2/UL primers)−Ct (amplification with UA1/UL primers).

For this example, it was determined that the sample: is homozygous for allele 1 if the ΔCt is greater than or equal to 4.5; homozygous for allele 2 if the ΔCt is less than or equal to −2; heterozygous if ΔCt is greater than or equal to −1 and less than or equal to 3.5; and no call is made if ΔCt is greater than −2 and less than −1 or greater than 3.5 and less than 4.5.

In this example, the ΔCt values were set for the genotype calls because, with the primers and assay conditions that were employed, the average ΔCt values for known heterozygotes are 1.25. One may set appropriate values for making genotype calls as appropriate by testing genomic DNA having known genotypes and determining appropriate values. In certain embodiments, for example, products with one of the allele specific primer-specific portions or its complement may result in more efficient PCR amplification than products with the other allele specific primer-specific portion or its complement. Accordingly, one may set the ΔCt values as appropriate for making genotype calls.

Performance data for the three different genomic DNAs for the three different SNPs tested in each assay in this example is shown in Table 4 below. The three different genomic DNAs were known collectively to exhibit all three possible genotypes for each locus. The genotype call (GT) that was determined in view of the Ct data is listed in Table 4 as “GT call.” The expected genotype that had been reported by Celera Genomics using TaqMan® assays is shown in Table 4 as “expected GT.”

TABLE 4

| | NA17140 | | | | |
|---|---|---|---|---|---|
| SNP | Ct(UA1/UL) | Ct(UA2/UL) | delta Ct | GT call | expected GT |
| Assay 1 | 31.10 | 40.00 | 8.90 | Hom 1 | Hom 1 |
| Assay 2 | 29.59 | 39.02 | 9.43 | Hom 1 | Hom 1 |
| Assay 3 | 32.21 | 38.40 | 6.19 | Hom 1 | Hom 1 |
| | NA17155 | | | | |
| SNP | Ct(UA1/UL) | Ct(UA2/UL) | delta Ct | GT call | expected GT |
| Assay 1 | 38.96 | 31.67 | −7.29 | Hom 2 | Hom 2 |
| Assay 2 | 37.65 | 30.88 | −6.77 | Hom 2 | Hom 2 |
| Assay 3 | 39.58 | 34.51 | −5.07 | Hom 2 | Hom 2 |
| | NA 17202 | | | | |
| SNP | Ct(UA1/UL) | Ct(UA2/UL) | delta Ct | GT call | expected GT |
| Assay 1 | 31.33 | 32.36 | 1.03 | het | het |
| Assay 2 | 31.07 | 32.32 | 1.25 | het | het |
| Assay 3 | 34.98 | 36.10 | 1.11 | het | het |

E. Proposed Modification To Procedure Above

In certain embodiments, the total ligation reaction volume may be less than 5 μL. In certain embodiments, certain robot pipetting may be employed. In certain embodiments, the genomic DNA in ligation reaction volume may be less than 10 ng/μL.

Although the invention has been described with reference to certain applications, methods, and compositions, it will be appreciated that various changes and modifications may be made without departing from the invention.

What is claimed is:

1. A method for detecting the presence or absence of at least one target nucleic acid sequence in a sample comprising:
forming a ligation reaction composition comprising the sample, and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion, wherein the 3′ primer-specific portion comprises a sequence, wherein the probes in each set are suitable for ligation together when hybridized adjacent to one another on a complementary target sequence;
forming a test composition by subjecting the ligation reaction composition to at least one cycle of ligation, wherein adjacently hybridizing complementary probes are ligated to one another to form a ligation product comprising the 5′ primer-specific portion, the target-specific portions, and the 3′ primer-specific portion;
forming at least one amplification reaction composition comprising:
at least a portion of the test composition;
a polymerase;
a double-stranded-dependent specific label, wherein the double-stranded-dependent label has a first detectable signal value when the double-stranded-dependent label is not exposed to double-stranded nucleic acid; and
at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product;
subjecting the at least one amplification reaction composition to at least one amplification reaction; and
detecting a second detectable signal value at least one of during and after the at least one amplification reaction, wherein a threshold difference between the first detectable signal value and the second detectable signal value indicates the presence of the target nucleic acid sequence, and wherein no threshold difference between the first detectable signal value and the second detectable signal value indicates the absence of the target nucleic acid sequence.

2. The method of claim 1, wherein:
the ligation reaction composition comprises:
at least two different probe sets for detecting at least two different target nucleic acid sequences, and wherein
a first probe set comprises (a) at least one first probe, comprising a target-specific portion that hybridizes to a first portion of a first target nucleic acid sequence and a 5′ primer-specific portion, wherein the 5′ primer-specific portion comprises a sequence and (b) at least one second probe, comprising a target-specific portion that hybridizes to a second portion of the first target nucleic acid sequence and a 3′ primer-specific portion, wherein the 3′ primer-specific portion comprises a sequence; and
a second probe set comprises (a) at least one first probe, comprising a target-specific portion that hybridizes to a first portion of a second target nucleic acid sequence, and a 5′ primer-specific portion, wherein the 5′ primer-specific portion comprises a sequence, and (b) at least one second probe, comprising a target-specific portion that hybridizes to a second portion of the second target nucleic acid sequence, and a 3′ primer-specific portion, wherein the 3′ primer-specific portion comprises a sequence;
wherein the sequence of the 5′ primer-specific portion of the first probe of the first probe set is different from the sequence of the 5′ primer-specific portion of the first probe of the second probe set and wherein the first target nucleic acid sequence is different from the second target nucleic acid sequence.

3. The method of claim 2:
wherein the forming of the at least one amplification reaction composition comprises forming at least two amplification reaction compositions comprising:
a first amplification reaction composition comprising:
at least a portion of the test composition;
a polymerase;
a double-stranded-dependent specific label, wherein the double-stranded-dependent label has a first detectable signal value when the double-stranded-dependent label is not exposed to double-stranded nucleic acid; and
a first primer set, the first primer set comprising (i) at least one first primer comprising the sequence of the 5′ primer-specific portion of the at least one first probe of the first probe set, and (ii) at least one second primer comprising a sequence complementary to the sequence of the 3′ primer-specific portion of the at least one second probe of the first probe set; and
a second amplification reaction composition comprising:
at least a portion of the test composition;
a polymerase;
a double-stranded-dependent label, wherein the double-stranded-dependent label has a first detectable signal value when the double-stranded-dependent label is not exposed to double-stranded nucleic acid; and
a second primer set, the second primer set comprising (i) at least one first primer comprising the sequence of the 5′ primer-specific portion of the at least one first probe of the second probe set, and
(ii) at least one second primer comprising a sequence complementary to the sequence of the 3′ primer-specific portion of the at least one second probe of the second probe set;
and wherein each of the at least two amplification reaction compositions are subjected to at least one amplification reaction;
and wherein the detecting comprises:
detecting a second detectable signal value at least one of during and after the at least one amplification reaction of the first amplification reaction composition, wherein a threshold difference between the first detectable signal value and the second detectable signal value of the at least one amplification reaction of the first amplification reaction composition indicates the presence of the first target nucleic acid sequence, and wherein no threshold difference between the first detectable signal value and the second detectable signal value of the at least one amplification reaction of the first amplification reaction composition indicates the absence of the first target nucleic acid sequence; and
detecting a second detectable signal value at least one of during and after the at least one amplification reaction of the second amplification reaction composition, wherein a threshold difference between the first detectable signal value and the second detectable signal value of the amplification reaction of the second amplification reaction composition indicates the presence of the second target nucleic acid sequence, and wherein no threshold difference between the first detectable signal value and the second detectable signal value of the at least one amplification reaction of the second amplification reaction composition indicates the absence of the second target nucleic acid sequence.

4. The method of claim 1, wherein:
the ligation reaction composition comprises:
at least two different probe sets for detecting at least two different target nucleic acid sequences, and wherein
a first probe set comprises (a) at least one first probe, comprising a target-specific portion that hybridizes to a first portion of a first target nucleic acid sequence and a 5′ primer-specific portion, wherein the 5′ primer-specific portion comprises a sequence and (b) at least one second probe, comprising a target-specific specific portion that hybridizes to a second portion of the first target nucleic acid sequence and a 3′ primer-specific portion, wherein the 3′ primer-specific portion comprises a sequence; and
a second probe set comprises (a) at least one first probe, comprising a target-specific portion that hybridizes to a first portion of a second target nucleic acid sequence, and a 5′ primer-specific portion, wherein the 5′ primer-specific portion comprises a sequence, and (b) at least one second probe, comprising a target-specific portion that hybridizes to a second portion of the second target nucleic acid sequence, and a 3′ primer-specific portion, wherein the 3′ primer-specific portion comprises a sequence;
wherein the sequence of the 3′ primer-specific portion of the second probe of the first probe set is different from the sequence of the 3′ primer-specific portion of the second probe of the second probe set and wherein the first target nucleic acid sequence is different from the second target nucleic acid sequence.

5. The method of claim 4:
wherein the forming of the at least one amplification reaction composition comprises forming at least two amplification reaction compositions comprising:
a first amplification reaction composition comprising:
at least a portion of the test composition;
a polymerase;
a double-stranded-dependent label, wherein the double-stranded-dependent label has a first detectable signal value when the double-stranded-dependent label is not exposed to double-stranded nucleic acid; and
a first primer set, the first primer set comprising (i) at least one first primer comprising the sequence of the 5′ primer-specific portion of the at least one first probe of the first probe set, and (ii) at least one second primer comprising a sequence complementary to the sequence of the 3′ primer-specific portion of the at least one second probe of the first probe set; and
a second amplification reaction composition comprising:
at least a portion of the test composition;
a polymerase;
a double-stranded-dependent label, wherein the double-stranded-dependent label has a first detectable signal value when the double-stranded-dependent label is not exposed to double-stranded nucleic acid; and
a second primer set, the second primer set comprising (i) at least one first primer comprising the sequence of the 5′ primer-specific portion of the at least one first probe of the second probe set, and
(ii) at least one second primer comprising a sequence complementary to the sequence of the 3′ primer-specific portion of the at least one second probe of the second probe set;
and wherein each of the at least two amplification reaction compositions are subjected to at least one amplification reaction;
and wherein the detecting comprises:
detecting a second detectable signal value at least one of during and after the at least one amplification reaction of the first amplification reaction composition, wherein a threshold difference between the first detectable signal value and the second detectable signal value of the at least one amplification reaction of the first amplification reaction composition indicates the presence of the first target nucleic acid sequence, and wherein no threshold difference between the first detectable signal value and the second detectable signal value of the at least one amplification reaction of the first amplification reaction composition indicates the absence of the first target nucleic acid sequence; and
detecting a second delectable signal value at least one of during and after the at least one amplification reaction of the second amplification reaction composition, wherein a threshold difference between the first detectable signal value and the second detectable signal value of the at least one amplification reaction of the second amplification reaction composition indicates the presence of the second target nucleic acid sequence, and wherein no threshold difference between the first detectable signal value and the second detectable signal value of the at least one amplification reaction of the second amplification reaction composition indicates the absence of the second target nucleic acid sequence.

6. The method of any one of claims 2 to 5, wherein the first target nucleic acid sequence and the second target nucleic acid sequence have different nucleotides at a given locus.

7. A method for detecting the presence or absence of at least one target nucleic acid sequence in a sample comprising:
forming a ligation reaction composition comprising the sample, and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion, wherein the 3′ primer-specific portion comprises a sequence, wherein the probes in each set are suitable for ligation together when hybridized adjacent to one another on a complementary target sequence;
forming a test composition by subjecting the ligation reaction composition to at least one cycle of ligation, wherein adjacently hybridizing complementary probes are ligated to one another to form a ligation product comprising the 5′ primer-specific portion, the target-specific portions, and the 3′ primer-specific portion;
forming at least one amplification reaction composition comprising:
at least a portion of the test composition,
a polymerase,
a double-stranded-dependent label; and
at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product;
subjecting the at least one amplification reaction composition to at least one amplification reaction; and
detecting the presence or absence of the target nucleic acid sequence by monitoring a signal at least one of during and after the at least one amplification reaction.

8. The method of claim 7:
wherein the detecting comprises determining a threshold cycle (Cₜ) value from the monitoring of the signal.

9. The method of claim 7:
wherein the detecting comprises determining a threshold time (Tₜ) value from the monitoring of the signal.

10. The method of claim 7, wherein:
the ligation reaction composition comprises:
at least two different probe sets for detecting at least two different target nucleic acid sequences, and wherein
a first probe set comprises (a) at least one first probe, comprising a target-specific portion that hybridizes to a first portion of a first target nucleic acid sequence and a 5′ primer-specific portion, wherein the 5′ primer-specific portion comprises a sequence and (b) at least one second probe, comprising a target-specific portion that hybridizes to a second portion of the first target nucleic acid sequence and a 3′ primer-specific portion, wherein the 3′ primer-specific portion comprises a sequence; and
a second probe set comprises (a) at least one first probe, comprising a target-specific portion that hybridizes to a first portion of a second target nucleic acid sequence, and a 5′ primer-specific portion, wherein the 5′ primer-specific portion comprises a sequence, and (b) at least one second probe, comprising a target-specific portion that hybridizes to a second portion of the second target nucleic acid sequence, and a 3′ primer-specific portion, wherein the 3′ primer-specific portion comprises a sequence;
wherein the sequence of the 5′ primer-specific portion of the first probe of the first probe set is different from the sequence of the 5′ primer-specific portion of the first probe of the second probe set and wherein the first target nucleic acid sequence is different from the second target nucleic acid sequence.

11. The method of claim 10:
wherein the forming of the at least one amplification reaction composition comprises forming at least two amplification reaction compositions comprising:
a first amplification reaction composition comprising:
at least a portion of the test composition;
a polymerase;
a double-stranded-dependent label; and
a first primer set, the first primer set comprising (i) at least one first primer comprising the sequence of the 5′ primer-specific portion of the at least one first probe of the first probe set, and (ii) at least one second primer comprising a sequence complementary to the sequence of the 3′ primer-specific portion of the at least one second probe of the first probe set; and
a second amplification reaction composition comprising:
at least a portion of the test composition;
a polymerase;
a double-stranded-dependent label; and
a second primer set, the second primer set comprising (i) at least one first primer comprising the sequence of the 5′ primer-specific portion of the at least one first probe of the second probe set, and
(ii) at least one second primer comprising a sequence complementary to the sequence of the 3′ primer-specific portion of the at least one second probe of the second probe set;
and wherein each of the at least two amplification reaction compositions are subjected to at least one amplification reaction;
and wherein the detecting comprises:
detecting the presence or absence of the first target nucleic acid sequence by monitoring a signal at least one of during and after the at least one amplification reaction of the first amplification reaction composition; and
detecting the presence or absence of the second target nucleic acid sequence by monitoring a signal at least one of during and after the at least one amplification reaction of the second amplification reaction composition.

12. The method of claim 11, wherein
the detecting of the presence or absence of the first target nucleic acid sequence comprises determining a first Cₜ value from the monitoring of the signal of the at least one amplification reaction of the first amplification reaction composition; and
the detecting of the presence or absence of the second target nucleic acid sequence comprises determining a second Cₜ value from the monitoring of the signal of the at least one amplification reaction of the second amplification reaction composition.

13. The method of claim 12, wherein the detecting of the presence or absence of the first target nucleic acid sequence and the second target nucleic acid sequence comprises comparing the first Cₜ value to the second Cₜ value.

14. The method of claim 12, wherein the first target nucleic acid sequence and the second target nucleic acid sequence have different nucleolides at a given locus, and the detecting of the presence or absence of the first target nucleic acid sequence and the second target nucleic acid sequence comprises comparing the first Cₜ value to the second Cₜ value.

15. The method of claim 11, wherein
the detecting of the presence or absence of the first target nucleic acid sequence comprises determining a first Tₜ value from the monitoring of the signal of the at least one amplification reaction of the first amplification reaction composition; and
the detecting of the presence or absence of the second target nucleic acid sequence comprises determining a second Tₜ value from the monitoring of the signal of the at least one amplification reaction of the second amplification reaction composition.

16. The method of claim 15, wherein the detecting of the presence or absence of the first target nucleic acid sequence and the second target nucleic acid sequence comprises comparing the first Tₜ value to the second Tₜ value.

17. The method of claim 15, wherein the first target nucleic acid sequence and the second target nucleic acid sequence have different nucleotides at a given locus, and the detecting of the presence or absence of the first target nucleic acid sequence and the second target nucleic acid sequence comprises comparing the first Tₜ value to the second Tₜ value.

18. The method of claim 7, wherein:
the ligation reaction composition comprises:
at least two different probe sets for detecting at least two different target nucleic acid sequences, and wherein
a first probe set comprises (a) at least one first probe, comprising a target-specific portion that hybridizes to a first portion of a first target nucleic acid sequence and a 5′ primer-specific portion, wherein the 5′ primer-specific portion comprises a sequence and (b) at least one second probe, comprising a target-specific portion that hybridizes to a second portion of the first target nucleic acid sequence and a 3′ primer-specific portion, wherein the 3′ primer-specific portion comprises a sequence; and
a second probe set comprises (a) at least one first probe, comprising a target-specific portion that hybridizes to a first portion of a second target nucleic acid sequence, and a 5′ primer-specific portion, wherein the 5′ primer-specific portion comprises a sequence, and (b) at least one second probe, comprising a target-specific portion that hybridizes to a second portion of the second target nucleic acid sequence, and a 3′ primer-specific portion, wherein the 3′ primer-specific portion comprises a sequence;
wherein the sequence of the 3′ primer-specific portion of the second probe of the first probe set is different from the sequence of the 3′ primer-specific portion of the second probe of the second probe set and wherein the first target nucleic acid sequence is different from the second target nucleic acid sequence.

19. The method of claim 18:
wherein the forming of the at least one amplification reaction composition comprises forming at least two amplification reaction compositions comprising:
a first amplification reaction composition comprising:
at least a portion of the test composition;
a polymerase;
a double-stranded-dependent; and
a first primer set, the first primer set comprising (i) at least one first primer comprising the sequence of the 5′ primer-specific portion of the at least one first probe of the first probe set, and (ii) at least one second primer comprising a sequence complementary to the sequence of the 3′ primer-specific portion of the at least one second probe of the first probe set; and
a second amplification reaction composition comprising:
at least a portion of the test composition;
a polymerase;
a double-stranded-dependent label; and
a second primer set, the second primer set comprising (i) at least one first primer comprising the sequence of the 5′ primer-specific portion of the at least one first probe of the second probe set, and
(ii) at least one second primer comprising a sequence complementary to the sequence of the 3′ primer-specific portion of the at least one second probe of the second probe set;
and wherein each of the at least two amplification reaction compositions are subjected to at least one amplification reaction;
and wherein the detecting comprises:
detecting the presence or absence of the first target nucleic acid sequence by monitoring a signal at least one of during and after the at least one amplification reaction of the first amplification reaction composition; and
detecting the presence or absence of the second target nucleic acid sequence by monitoring a signal at least one of during and after the at least one amplification reaction of the second amplification reaction composition.

20. The method of claim 19, wherein
the detecting of the presence or absence of the first target nucleic acid sequence comprises determining a first Cₜ value from the monitoring of the signal of the at least one amplification reaction of the first amplification reaction composition; and
the detecting of the presence or absence of the second target nucleic acid sequence comprises determining a second Cₜ value from the monitoring of the signal of the at least one amplification reaction of the second amplification reaction composition.

21. The method of claim 20, wherein the detecting of the presence or absence of the first target nucleic acid sequence and the second target nucleic acid sequence comprises comparing the first Cₜ value to the second Cₜ value.

22. The method of claim 20, wherein the first target nucleic acid sequence and the second target nucleic acid sequence have different nucleotides at a given locus, and the detecting of the presence or absence of the first target nucleic acid sequence and the second target nucleic acid sequence comprises comparing the first Cₜ value to the second Cₜ value.

23. The method of claim 19, wherein
the detecting of the presence or absence of the first target nucleic acid sequence comprises determining a first Tₜ value from the monitoring of the signal of the at least one amplification reaction of the first amplification reaction composition; and
the detecting of the presence or absence of the second target nucleic acid sequence comprises determining a second Tₜ value from the monitoring of the signal of the at least one amplification reaction of the second amplification reaction composition.

24. The method of claim 23, wherein the detecting of the presence or absence of the first target nucleic acid sequence and the second target nucleic acid sequence comprises comparing the first Tₜ value to the second Tₜ value.

25. The method of claim 23, wherein the first target nucleic acid sequence and the second target nucleic acid sequence have different nucleotides at a given locus, and the detecting of the presence or absence of the first target nucleic acid sequence and the second target nucleic acid sequence comprises comparing the first Tₜ value to the second Tₜ value.

26. The method of any one of claims 10 to 12, 15, 18 to 20, and 23, wherein the first target nucleic acid sequence and the second target nucleic acid sequence have different nucleotides at a given locus.

27. A method for detecting the presence or absence of at least one target nucleic acid sequence in a sample comprising:
(a) forming at least one reaction composition comprising:
the sample;
a ligation probe set for the target nucleic acid sequence, the probe set comprising (a) at least one first probe, comprising a target-specific portion and a 5′ primer-specific portion, wherein the 5′ primer-specific portion comprises a sequence and (b) at least one second probe, comprising a target-specific portion and a 3′ primer-specific portion, wherein the 3′ primer-specific portion comprises a sequence, wherein the probes in each set are suitable for ligation together when hybridized adjacent to one another on a complementary target sequence;
a polymerase;
a double-stranded-dependent label, wherein the double-stranded-dependent label has a first detectable signal value when the double-stranded-dependent label is not exposed to double-stranded nucleic acid; and
at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product;
(b) subjecting the reaction composition to at least one cycle of ligation, wherein adjacently hybridizing complementary probes are ligated to one another to form a ligation product comprising the 5′ primer-specific portion, the target-specific portions, and the 3′ primer-specific portion;
(c) after the at least one cycle of ligation, subjecting the reaction composition to at least one amplification reaction; and
(d) detecting a second detectable signal value at least one of during and after the at least one amplification reaction, wherein a threshold difference between the first detectable signal value and the second detectable signal value indicates the presence of the target nucleic acid sequence, and wherein no threshold difference between the first detectable signal value and the second detectable signal value indicates the absence of the target nucleic acid sequence.

28. The method of claim 27:
wherein the forming of the at least one reaction composition comprises forming at least two reaction compositions for detecting at least two different target nucleic acid sequences, the at least two reaction compositions comprising:
a first reaction composition comprising:
a first probe set comprises (a) at least one first probe, comprising a target-specific portion that hybridizes to a first portion of a first target nucleic acid sequence and a 5′ primer-specific portion, wherein the 5′ primer-specific portion comprises a sequence and (b) at least one second probe, comprising a target-specific portion that hybridizes to a second portion of the first target nucleic acid sequence and a 3′ primer-specific portion, wherein the 3′ primer-specific portion comprises a sequence;
a polymerase;
a double-stranded-dependent label, wherein the double-stranded-dependent label has a first delectable signal value when the double-stranded-dependent label is not exposed to double-strnded nucleic acid; and
a first primer set, the first primer set comprising (i) at least one first primer comprising the sequence of the 5′ primer-specific portion of the at least one first probe of the first probe set, and (ii) at least one second primer comprising a sequence complementary to the sequence of the 3′ primer-specific portion of the at least one second probe of the first probe set; and
a second amplification reaction composition comprising:
a second probe set comprises (a) at least one first probe, comprising a target-specific portion that hybridizes to a first portion of a second target nucleic acid sequence, and a 5′ primer-specific portion, wherein the 5′ primer-specific portion comprises a sequence, and (b) at least one second probe, comprising a target-specific portion that hybridizes to a second portion of the second target nucleic acid sequence, and a 3′ primer-specific portion, wherein the 3′ primer-specific portion comprises a sequence;
a polymerase;
a double-stranded-dependent label, wherein the double-stranded-dependent label has a first detectable signal value when the double-stranded-dependent label is not exposed to double-stranded nucleic acid; and
a second primer set, the second primer set comprising (i) at least one first primer comprising the sequence of the 5′ primer-specific portion of the at least one first probe of the second probe set, and
(ii) at least one second primer comprising a sequence complementary to the sequence of the 3′ primer-specific portion of the at least one second probe of the second probe set;
wherein the first target nucleic acid sequence is different from the second target nucleic acid sequence; and
wherein each of the at least two amplification reaction compositions are subjected to at least one amplification reaction;
and wherein the detecting comprises:
detecting a second detectable signal value at least one of during and after the at least one amplification reaction of the first amplification reaction composition, wherein a threshold difference between the first detectable signal value and the second detectable signal value of the at least one amplification reaction of the first amplification reaction composition indicates the presence of the first target nucleic acid sequence, and wherein no threshold difference between the first detectable signal value and the second detectable signal value of the at least one amplification reaction of the first amplification reaction composition indicates the absence of the first target nucleic acid sequence; and
detecting a second detectable signal value at least one of during and after the at least one amplification reaction of the second amplification reaction composition, wherein a threshold difference between the first detectable signal value and the second detectable signal value of the at least one amplification reaction of the second amplification reaction composition indicates the presence of the second target nucleic acid sequence, and wherein no threshold difference between the first detectable signal value and the second detectable signal value of the at least one amplification reaction of the second amplification reaction composition indicates the absence of the second target nucleic acid sequence.

29. The method of claim 28, wherein the sequence of the 5′ primer-specific portion of the first probe of the first probe set is the same as the sequence of the 5′ primer-specific portion of the first probe of the second probe set.

30. The method of claim 29, wherein the sequence of the 3′ primer-specific portion of the first probe of the first probe set is the same as the sequence of the 3′ primer-specific portion of the first probe of the second probe set.

31. The method of claim 28, wherein the sequence of the 3′ primer-specific portion of the first probe of the first probe set is the same as the sequence of the 3′ primer-specific portion of the first probe of the second probe set.

32. The method of any one of claims 28 to 31, wherein the first target nucleic acid sequence and the second target nucleic acid sequence have different nucleotides at a given locus.

33. A method for detecting the presence or absence of at least one target nucleic acid sequence in a sample comprising:
(a) forming at least one reaction composition comprising:
the sample;
a ligation probe set for the target nucleic acid sequence, the probe set comprising (a) at least one first probe, comprising a target-specific portion and a 5′ primer-specific portion, wherein the 5′ primer-specific portion comprises a sequence and (b) at least one second probe, comprising a target-specific portion and a 3′ primer-specific portion, wherein the 3′ primer-specific portion comprises a sequence, wherein the probes in each set are suitable for ligation together when hybridized adjacent to one another on a complementary target sequence;
a polymerase;
a double-stranded-dependent label; and
at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product;
(b) subjecting the reaction composition to at least one cycle of ligation, wherein adjacently hybridizing complementary probes are ligated to one another to form a ligation product comprising the 5′ primer-specific portion, the target-specific portions, and the 3′ primer-specific portion;
(c) after the at least one cycle of ligation, subjecting the reaction composition to at least one amplification reaction; and
(d) detecting the presence or absence of the target nucleic acid sequence by monitoring a signal at least one of during and after the at least one amplification reaction.

34. The method of claim 33:
wherein the detecting comprises determining a threshold cycle (Cₜ) value from the monitoring of the signal.

35. The method of claim 33:
wherein the detecting comprises determining a threshold time (Tₜ) value from the monitoring of the signal.

36. The method of claim 33:
wherein the forming of the at least one reaction composition comprises forming at least two reaction compositions for detecting at least two different target nucleic acid sequences, the at least two reaction compositions comprising:
a first reaction composition comprising:
a first probe set comprises (a) at least one first probe, comprising a target-specific portion that hybridizes to a first portion of a first target nucleic acid sequence and a 5′ primer-specific portion, wherein the 5′ primer-specific portion comprises a sequence and (b) at least one second probe, comprising a target-specific portion that hybridizes to a second portion of the first target nucleic acid sequence and a 3′ primer-specific portion, wherein the 3′ primer-specific portion comprises a sequence;
a polymerase;
a double-stranded-dependent; and
a first primer set, the first primer set comprising (i) at least one first primer comprising the sequence of the 5′ primer-specific portion of the at least one first probe of the first probe set, and (ii) at least one second primer comprising a sequence complementary to the sequence of the 3′ primer-specific portion of the at least one second probe of the first probe set; and
a second reaction composition comprising:
a second probe set comprises (a) at least one first probe, comprising a target-specific portion that hybridizes to a first portion of a second target nucleic acid sequence, and a 5′ primer-specific portion, wherein the 5′ primer-specific portion comprises a sequence, and (b) at least one second probe, comprising a target-specific portion that hybridizes to a second portion of the second target nucleic acid sequence, and a 3′ primer-specific portion, wherein the 3′ primer-specific portion comprises a sequence;
a polymerase;
a double-stranded-dependent label; and
a second primer set, the second primer set comprising (i) at least one first primer comprising the sequence of the 5′ primer-specific portion of the at least one first probe of the second probe set, and (ii) at least one second primer comprising a sequence complementary to the sequence of the 3′ primer-specific portion of the at least one second probe of the second probe set;
wherein the first target nucleic acid sequence is different from the second target nucleic acid sequence; and
wherein each of the at least two reaction compositions are subjected to at least one amplification reaction;
and wherein the detecting comprises:
detecting the presence or absence of the first target nucleic acid sequence by monitoring a signal at least one of during and after the at least one amplification reaction of the first reaction composition; and
detecting the presence or absence of the second target nucleic acid sequence by monitoring a signal at least one of during and after the at least one amplification reaction of the second reaction composition.

37. The method of claim 36, wherein
the detecting of the presence or absence of the first target nucleic acid sequence comprises determining a first Cₜ value from the monitoring of the signal of the at least one amplification reaction of the first reaction composition; and
the detecting of the presence or absence of the second target nucleic acid sequence comprises determining a second Cₜ value from the monitoring of the signal of the at least one amplification reaction of the second reaction composition.

38. The method of claim 36, wherein the first target nucleic acid sequence and the second target nucleic acid sequence have different nucleotides at a given locus.

39. The method of claim 37, wherein the first target nucleic acid sequence and the second target nucleic acid sequence have different nucleotides at a given locus.

40. The method of claim 37, wherein the detecting of the presence or absence of the first target nucleic acid sequence and the second target nucleic acid sequence comprises comparing the first Cₜ value to the second Cₜ value.

41. The method of claim 37, wherein the first target nucleic acid sequence and the second target nucleic acid sequence have different nucleotides at a given locus, and the detecting of the presence or absence of the first target nucleic acid sequence and the second target nucleic acid sequence comprises comparing the first Cₜ value to the second Cₜ value.

42. The method of claim 36, wherein the detecting of the presence or absence of the first target nucleic acid sequence comprises determining a first Tₜ value from the monitoring of the signal of the at least one amplification reaction of the first reaction composition; and
the detecting of the presence or absence of the second target nucleic acid sequence comprises determining a second Tₜ value from the monitoring of the signal of the at least one amplification reaction of the second reaction composition.

43. The method of claim 42, wherein the first target nucleic acid sequence and the second target nucleic acid sequence have different nucleotides at a given locus.

44. The method of claim 42, wherein the detecting of the presence or absence of the first target nucleic acid sequence and the second target nucleic acid sequence comprises comparing the first Tₜ value to the second Tₜ value.

45. The method of claim 42, wherein the first target nucleic acid sequence and the second target nucleic acid sequence have different nucleotides at a given locus, and the detecting of the presence or absence of the first target nucleic acid sequence and the second target nucleic acid sequence comprises comparing the first Tₜ value to the second Tₜ value.

46. The method of any one of claims 36 to 45, wherein the sequence of the 5′ primer-specific portion of the first probe of the first probe set is the same as the sequence of the 5′ primer-specific portion of the first probe of the second probe set.

47. The method of claim 46, wherein the sequence of the 3′ primer-specific portion of the first probe of the first probe set is the same as the sequence of the 3′ primer-specific portion of the first probe of the second probe set.

48. The method of any one of claims 36 to 45, wherein the sequence of the 3′ primer-specific portion of the first probe of the first probe set is the same as the sequence of the 3′ primer-specific portion of the first probe of the second probe set.

49. A kit for detecting at least one target nucleic acid sequence in a sample comprising:
(a) a ligation probe set for each target nucleic acid sequence, the probe set comprising
(i) at least one first probe, comprising a target-specific portion, a 5′ primer-specific portion, wherein the 5′ primer-specific portion comprises a sequence, and
(ii) at least one second probe, comprising a target-specific portion, a 3′ primer-specific portion, wherein the 3′ primer-specific portion comprises a sequence,
wherein the probes in each set are suitable for ligation together when hybridized adjacent to one another on a complementary target nucleic acid sequence; and
(b) a double-stranded-dependent label.

50. The kit of claim 49, further comprising at least one primer set comprising (i) at least one first primer comprising the sequence of the 5′ primer-specific portion of the at least one first probe, and (ii) at least one second primer comprising a sequence complementary to the sequence of the 3′ primer-specific portion of the at least one second probe.

51. A method for detecting the presence or absence of at least one target nucleic acid sequence in a sample comprising:
forming a ligation reaction composition comprising the sample; a ligation probe set for each target nucleic acid sequence, the probe set comprising (a) at least one first probe, comprising a target-specific portion, and (b) at least one second probe, comprising a target-specific portion, wherein the probes in each set are suitable for ligation together when hybridized adjacent to one another on a complementary target sequence; and poly-deoxy-inosinic-deoxy-cytidylic acid;
forming a test composition by subjecting the ligation reaction composition to at least one cycle of ligation, wherein adjacently hybridizing complementary probes are ligated to one another to form a ligation product comprising the 5′ primer-specific portion, the target-specific portions, and the 3′ primer-specific portion; and
detecting the presence or absence of the ligation product to detect the presence or absence of the at least one target nucleic acid sequence.

52. A method for detecting the presence or absence of at least one target nucleic acid sequence in a sample comprising:
forming a ligation reaction composition comprising the sample, poly-deoxy-inosinic-deoxy-cytidylic acid, and a ligation probe set for each target nucleic acid sequence, the probe set comprising (a) at least one first probe, comprising a target-specific portion and a 5′ primer-specific portion, wherein the 5′ primer-specific portion comprises a sequence, and (b) at least one second probe, comprising a target-specific portion and a 3′ primer-specific portion, wherein the 3′ primer-specific portion comprises a sequence, wherein the probes in each set are suitable for ligation together when hybridized adjacent to one another on a complementary target sequence;
forming a test composition by subjecting the ligation reaction composition to at least one cycle of ligation, wherein adjacently hybridizing complementary probes are ligated to one another to form a ligation product comprising the 5′ primer-specific portion, the target-specific portions, and the 3′ primer-specific portion;
forming at least one amplification reaction composition comprising:
at least a portion of the test composition;
a polymerase; and
at least one primer set, the primer set comprising (i) at least one first primer comprising the sequence of the 5′ primer-specific portion of the ligation product, and (ii) at least one second primer comprising a sequence complementary to the sequence of the 3′ primer-specific portion of the ligation product;
subjecting the at least one amplification reaction composition to at least one amplification reaction; and
detecting the presence or absence of the target nucleic acid sequence by detecting whether the at least one amplification reaction results in amplification product from ligation product.

53. A kit for detecting at least one target nucleic acid sequence in a sample comprising:
(a) a ligation probe set for each target nucleic acid sequence, the probe set comprising
(i) at least one first probe, comprising a target-specific portion, a 5′ primer-specific specific portion, wherein the 5′ primer-specific portion comprises a sequence, and
(ii) at least one second probe, comprising a target-specific portion, a 3′ primer-specific portion, wherein the 3′ primer-specific portion comprises a sequence,
wherein the probes in each set are suitable for ligation together when hybridized adjacent to one another on a complementary target nucleic acid sequence; and
(b) a buffer comprising poly-deoxy-inosinic-deoxy-cytidylic acid.

54. The kit of claim 53, further comprising at least one primer set comprising (i) at least one first primer comprising the sequence of the 5′ primer-specific portion of the at least one first probe, and (ii) at least one second primer comprising a sequence complementary to the sequence of the 3′ primer-specific portion of the at least one second probe.

55. A composition for a ligation reaction comprising a ligase and poly-deoxy-inosinic-deoxy-cytidylic acid.


SEQUENCE LISTING (14 sequences)

SEQ ID NO: 1 - DNA, 15 bp, Artificial Sequence
  Description of Artificial Sequence Synthetic probe
catgccaatgacgga

SEQ ID NO: 2 - DNA, 15 bp, Artificial Sequence
  Description of Artificial Sequence Synthetic probe
catgcgaatgacggc

SEQ ID NO: 3 - DNA, 34 bp, Artificial Sequence
  Description of Artificial Sequence Synthetic probe
tgatgctactggatcgctgaaagcacattcctcg

SEQ ID NO: 4 - DNA, 32 bp, Artificial Sequence
  Description of Artificial Sequence Synthetic probe
ttgcctgctcgacttagaaagcacattcctca

SEQ ID NO: 5 - DNA, 63 bp, Artificial Sequence
  Description of Artificial Sequence Synthetic probe
gtctttgttaagtgcaggagcgcaaatccgtatagccaaagtggtatcactggatagcgacgt

SEQ ID NO: 6 - DNA, 34 bp, Artificial Sequence
  Description of Artificial Sequence Synthetic probe
tgatgctactggatcgctgcccatacactgagac

SEQ ID NO: 7 - DNA, 34 bp, Artificial Sequence
  Description of Artificial Sequence Synthetic probe
ttgcctgctcgacttagagcccatacactgagat

SEQ ID NO: 8 - DNA, 61 bp, Artificial Sequence
  Description of Artificial Sequence Synthetic probe
gctccatattgatttatttccgagtcggacaatcctgcgttacatcactggatagcgacgt

SEQ ID NO: 9 - DNA, 42 bp, Artificial Sequence
  Description of Artificial Sequence Synthetic probe
tgatgctactggatcgctagctttaaaacattttgttgtata

SEQ ID NO: 10 - DNA, 40 bp, Artificial Sequence
  Description of Artificial Sequence Synthetic probe
ttgcctgctcgacttagactttaaaacattttgttgtatg

SEQ ID NO: 11 - DNA, 58 bp, Artificial Sequence
  Description of Artificial Sequence Synthetic probe
tagttcagatcttgtaatagattgccaccttggaactgcgatcactggatagcgacgt

SEQ ID NO: 12 - DNA, 18 bp, Artificial Sequence
  Description of Artificial Sequence Primer
tgatgctactggatcgct

SEQ ID NO: 13 - DNA, 18 bp, Artificial Sequence
  Description of Artificial Sequence Primer
ttgcctgctcgacttaga

SEQ ID NO: 14 - DNA, 18 bp, Artificial Sequence
  Description of Artificial Sequence Primer
acgtcgctatccagtgat